(19) United States
(12) Patent Application Publication
(10) Pub. No.: US 2008/0057136 A1
(43) Pub. Date: Mar. 6, 2008

(54) Disinfecting Composition and Methods of Making and Using Same

(75) Inventors: Victor S. Polyakov, Moscow (RU); Valeriy V. Ermilov, Moscow (RU); Vladimir S. Kuzmin, Moscow (RU); Oleg I. Lukashov, Moscow (RU)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Veckis Industries Ltd., Vaduz (LI)

(21) Appl. No.: 10/578,961

(22) PCT Filed: Nov. 11, 2004
(86) PCT No.: PCT/IB04/03685

§ 371 Date: Aug. 16, 2007

Related U.S. Application Data
(60) Provisional application No. 60/518,891, filed on Nov. 11, 2003.
(60) Provisional application No. 60/523,190, filed on Nov. 13, 2003.

(51) Int. Cl. A61K 31/195; A61K 31/40; A61K 31/415; A61K 31/66; A61K 33/04; A61K 33/24; A61K 33/26; A61K 33/28; A61K 33/32; A61K 33/34; A61K 33/36; A61P 17/00

(52) U.S. Cl. 424/620; 424/630; 424/639; 424/641; 424/644; 424/646; 424/649; 424/650; 424/652; 424/654; 424/702; 514/399; 514/419; 514/561; 514/562; 514/564; 514/567; 514/75

(57) ABSTRACT

The invention relates to antimicrobial compositions that can be used for disinfecting and can be applied in various aspects of the national economy, medicine, and laboratories of all types. The antimicrobial compositions comprise a chelating metal complex compound with a monodentate bidentate or polydentate ligand that exhibits affinity to hydrogen ion, an ionogenic surfactant, and a solvent. The compositions of the invention display antiseptic properties. The antimicrobial compositions are active against Gram-positive and Gram-negative bacteria, fungi, viruses, and spores, and can be applied in a broad temperature interval. Methods of using the compositions of the present invention in the treatment and prevention of diseases caused by a variety of pathogens are further provided.

### FIELD OF THE INVENTION

The present invention relates to microbiocidal and sporicidal compositions, and more particularly antiviral, antibacterial and antifungal compositions for use alone or in combination with other chemical elements. These compositions are useful in the prevention or treatment of infections caused by pathogens.

### BACKGROUND OF THE INVENTION

One of the well known disinfecting agents is hydrogen peroxide and preparations thereof. A representative of this group is a disinfecting preparation containing hydrogen peroxide, magnesium laurylsulphate, glycerin, sodium oleate, the disodium salt of EDTA, sodium benzoate and water (RU2108810 C1, 1998). This agent is intended for decontaminating surfaces in houses, sanitary appliances, linen, medical goods. It is not toxic to humans or animals, but-it is not sufficiently efficacious.

Broadly known in the art are bactericidal compositions exhibiting an increased activity containing lanthionine and a chelating agent. The suitable chelating agents are, for example, ethylenediaminotetraacetic acid (EDTA), its salts and citrate (see, e.g., U.S. Pat. Nos. 5,260,271 and 5,334,582).

A bactericide and disinfectant comprising a metal complex with an α-amino acid in an acidic medium is also known (U.S. Pat. No. 6,242,009).

It is known that chelating metal complexes exist in an acidic medium only in negligible concentrations (Fundamentals of Analytical Chemistra Book 1, Moscow—“Mir”—D. Skoog, D. West, 1979). For example, a chelating agent such as EDTA completely binds metal ions to form chelating complexes at a pH above 6.0. For weaker chelating agents, of which natural amino acids are an example, the pH values of the media should not be higher in order to completely bind all metal ions into chelating complexes. The investigations carried out by the inventors have revealed that in U.S. Pat. No. 6,242,009 (the -“009 patent”) transformation of amino acids and metal ions into nondissociating chelating complexes can occur only at a pH >7.0, and addition of mineral acids in accordance with the examples cited in the patent leads to the destruction of the chelating complexes. In addition, the amino group of the amino acid is protonated, and the metal exists in an ionic form. The antimicrobial activity of the compounds cited in '009 can be attributed not to the activity of chelating complexes but to metal ions, which, as is known from the literature, also exhibit certain bactericidal activity, in particular, the cited silver ions. It should also be noted that arsenic and selenium compounds are cited in the '009 patent as metals and their antibacterial activity can be attributed to a high toxicity to all living organisms, including humans. There is no doubt that the presence of strong disinfectants (chlorohexydine, hydrogen peroxide), which are introduced as additives to the complexes cited in '009 patent, can increase the activity of the preparation.

Also described in the literature are bactericidal compositions that include cetyltrimethylammonium chloride as an active compound (DE 4326866, 1995; U.S. Pat. No. 5,206,016; U.S. Pat. No. 5,575,991). Of interest is an antiseptic preparation that includes as an active compound cetyltrimethylammonium chloride, a mineral or an organic acid and a solvent (RU 2118174 C1). The known compound exhibits bactericidal activity towards gram negative microflora, and it is not substantially effective towards intestinal and other bacterial and viral infections, including anthrax.

A disinfecting preparation containing bacteriocine, a chelating agent, a stabilizer, a surfactant, a salt, and an alcohol (RU 2163145) is also known in the art. The known preparation is used for impregnating napkins that are applied for prophylaxis of mastitis in animals.

A composition related to the present invention is a disinfecting preparation that contains a peroxide compound, a surfactant, a chelating complex and a solvent (RU20614497). This composition is active only when used at positive temperatures of 18-25° C. The time of the bacteria inactivation is varied in the interval of 5-30 minutes.

Antipathogenic compositions and methods that decrease the infectivity, morbidity, and mortality associated with pathogenic exposure are needed. Such compositions and methods should preferably not have undesirable properties of promoting microbial resistance or of being toxic to the recipient.

### SUMMARY OF THE INVENTION

The object of the present invention is to provide a highly effective universal disinfecting, antiseptic and bactericidal, fungicidal or viricidal composition, which is useful in a broad range of positive and negative temperatures and in increasing the length of microbiocidal and disinfectant action. A further objective of the invention is to enhance the length of time of the microbiocidal or disinfectant action. The present composition is suitable for long-term storage, is safe, exhibits high bactericidal, viricidal, fungicidal, and sporocidal activity, and is nontoxic to animals and humans. The present antimicrobial and sporicidal compositions are useful in a wide variety of applications. These compositions are useful as topical formulations in the treatment of microbial infections in a subject. The present compositions can be applied to various surfaces, and, when so applied, these compositions serve as sterilizers or sanitizers. Similarly, the present compositions can be used in application areas such as, for example, as a disinfectant in swimming pools, spas, etc., as a laundry soap or detergent additive, as a paint or surface coating additive, as a natural or synthetic surface preservative such as for the prevention of microfloral growth on surfaces (e.g., polymers, plastics or wood), and as a hard surface or carpet sanitizer. These compositions are generally useful in control and/or elimination of microflora and spores in many industrial, medical, agricultural, veterinary and domestic applications. Additionally, the present compositions can be employed to sterilize or disinfect gaseous environments including, for example, the cleansing of the atmosphere in homes and industrial sites, as well as in airplanes, etc.

### BRIEF DESCRIPTION OF THE DRAWINGS

FIG. 1 summarizes the time-kill analysis of S. aureus challenged with test compositions.

FIG. 2 summarizes the time-kill analysis of P. aeruginosa challenged with test compositions.

FIG. 3 summarizes the time-kill analysis of E. coli challenged with test compositions.

FIG. 4 summarizes the time-kill analysis of T. rubrum challenged with test compositions.

FIG. 5 summarizes the time kill analysis of C. albicans challenged with test compositions.

FIG. 6 summarizes the time-kill analysis of B. subtilis challenged with test compositions.

FIG. 7 summarizes the neutralizer effectiveness control and confirmation counts results for the time-kill analysis of S. aureus, E. coli, and T. rubrum.

### DETAILED DESCRIPTION OF THE INVENTION

### I. Definitions

As used herein the term “microorganism” refers to microscopic organisms and taxonomically related macroscopic organisms within the categories of algae, bacteria, fungi (including lichens), protozoa, viruses, and subviral agents. The term microorganism encompasses both those organisms that are in and of themselves pathogenic to another organism (e.g., animals, including humans, and plants) and those organisms that produce agents that are pathogenic to another organism, while the organism itself is not directly pathogenic or ineffective to other organisms.

As used herein the term “pathogen,” and grammatical equivalents, refers to an organism, including microorganisms, that causes disease in another organism (e.g., animals and plants) by directly infecting the other organism or by producing agents that causes disease in another organism (e.g., bacteria that produce pathogenic toxins and the like).

The terms “host” or “subject,” as used herein, refer to organisms to be treated by the compositions of the present invention. Such organisms include organisms that are exposed to, or suspected of being exposed to, one or more pathogens. Such organisms also include organisms to be treated so as to prevent undesired exposure to pathogens. Organisms include, but are not limited to animals (e.g., humans, domesticated animal species, wild animals) and plants.

As used herein, the term “inactivating,” and grammatical equivalents, means having the ability to kill, eliminate or reduce the capacity of a pathogen to infect and/or cause a pathological response in a host.

As used herein, the terms “contacted” and “exposed” refer to bringing one or more of the compositions of the present invention into contact with a pathogen or a sample to be protected against pathogens such that the compositions of the present invention may inactivate the microorganism or pathogenic agents, if present. The present invention may inactivate the microorganism or pathogenic agents, if present. The present invention contemplates that the disclosed compositions are contacted with the pathogens or microbial agents in sufficient volumes and/or concentrations to inactivate the pathogens or microbial agents.

As used herein, the term “topically active agents” refers to compositions of the present invention that elicit pharmacological responses at the site of application (contact) to a host.

As used herein, the term “surface” is used in its broadest sense. In one sense, the term refers to the outermost boundaries of an organism or inanimate object (e.g., vehicles, buildings and food processing equipment, etc.) that are capable of being contacted by the compositions of the present invention (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions of the invention by any of a number of transdermal delivery routes (e.g., injection, ingestion, transdermal delivery, inhalation, and the like).

As used herein, “pathogenic microbes or microorganisms” is intended to include pathogenic bacteria, fungi, viruses, etc. that do not normally reside in the host or that have over populated in the host to a pathogenic degree.

As used herein, a “microbiocidal composition” is a composition of the present invention that inhibits bacterial, yeast, fungal, or viral activation and/or proliferation.

As used herein, a “sporicidal composition” is a composition of the present invention that inhibits bacterial, yeast, or fungal spore activation and/or proliferation.

As used herein, “dosage” or “dosage unit form” refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of preparing such an active compound for the treatment of individuals.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like). A subject can also be a plant.

An “effective amount” of the microbiocidal or sporicidal compositions, as used herein, is a quantity sufficient to achieve a desired therapeutic and/or prophylactic effect, for example, an amount which results in the prevention of or a decrease in the symptoms associated with a disease or disorder that is being treated, e.g., the diseases associated with bacterial, viral, yeast or other fungal infection. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the microbiocidal or sporicidal compositions of the present invention sufficient for achieving a therapeutic or prophylactic effect, ranges from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day.

Alternatively, an effective amount of the microbiocidal or sporicidal compositions of the present invention sufficient for achieving a therapeutic or prophylactic effect, range from about 0.000001 mg per cm² of surface per day to about 10,000 mg per cm² of surface per day. Preferably, the dosage ranges are from about 0.0001 mg per cm² of surface per day to about 100 mg per cm² of surface per day.

As used herein, the term “inactivating,” and grammatical equivalents, means having the ability to kill, eliminate or reduce the capacity of a pathogen to infect and/or cause a pathological response in a host.

The microbiocidal or sporicidal compositions of the present invention can be administered in combination with one or more additional therapeutic compounds.

### II. General

All references cited in this application are expressly incorporated herein by reference hereto. It will be understood by those skilled in the art that various modifications and substitutions may be made to the invention as described above without departing from the spirit and scope of the invention. Accordingly, it is understood that the present invention has been described by way of illustration and not limitation.

The present invention is directed to an antiviral, antibacterial, and antifungal composition for use in a wide range of products. The composition is biologically active against a broad spectrum of viruses, bacteria, fungi, and other pathenogenic species. Specifically, the composition of the present invention kills viruses, bacteria and fungi. The biologically active composition of the present invention includes a chelating metal complex compound with a monodentate, bidentate, or polydentate ligand that exhibits affinity to hydrogen ion, an ionogenic surfactant, and a solvent.

The present invention further relates to compositions and methods for decreasing the infectivity, morbidity, and rate of mortality associated with a variety of pathogens, as well as to methods and compositions for decontaminating areas, samples, solutions, and foodstuffs colonized or otherwise infected by pathogens and microorganisms. Specifically, the present invention provides a novel composition having excellent antiviral, antibacterial and antifungal properties to provide protection against a broad spectrum of potential pathogens. Further, the composition of the present invention can be used in combination with other chemical agents including hydrophilic compounds and hydrophobic polymers, as specifically set forth in previous patent application Ser. No. 106,513, now U.S. Pat. No. 5,417,968, to provide such products as a prophylactic skin barrier providing antiviral, antibacterial and antifungal protection.

In some embodiments, the present invention provides compositions and methods suitable for treating animals, including humans, exposed to pathogens or the threat of pathogens. In some embodiments, the animal is contacted with effective amounts of the compositions prior to exposure to pathogenic organisms. In other embodiments, the animal is contacted with effective amounts of the compositions after exposure to pathogenic organisms. Thus, the present invention contemplates both the prevention and treatment of microbiological infections. When used in antiseptic applications, the methods and compositions of the invention can be used to treat a broad spectrum of infections by pathogenic microbes, preferably with a minimum of damage to normal flora.

Buffering of the microbiocidal or sporicidal composition provides for the desirable bactericidal effect at all pH values of human skin. The pH value of the preparation is weakly alkaline, i.e. about 7.6±0.5. The compositions find use in prophylaxis and in disinfection of contaminated open areas of human and animal skin, as well as of the surfaces of various materials. By its content and principal of action, the preparation is safe for humans and animals, is nontoxic, does not irritate skin, is chemically neutral towards all construction materials and fabrics made of natural and synthetic fibers, and does not cause corrosion of metals. The microbiocidal or sporicidal composition kills 99.99% of microbes and spores. By acute toxicity, the preparation is related to the IY class of low hazard compounds.

If the composition of the present invention is applied over skin, hair, nails or mucous membranes, the bactericidal or sporicidal effect is retained for not less than 2 h. The temperature range for skin application of the microbiocidal or sporicidal compositions is from about −20° C. to about +40° C. to about +50° C.

A mixture of effective amounts of ingredients exhibits a synergistic effect, and the disinfecting properties are increased by combining the active ingredients.

In other embodiments, the present invention provides compositions and methods suitable for decontaminating areas, solutions and surfaces, including organic and inorganic samples that are exposed to pathogens or suspected of containing pathogens. In still other embodiments of the present invention, the compositions are used as additives to prevent the growth of harmful or undesired microorganisms in biological and environmental samples.

If the composition of the present invention is applied over the surface of materials, fabrics, or protective coverings the bactericidal or sporicidal effect is retained for at least 24 h. The temperature range for surface application of the microbiocidal or sporicidal compositions is from about −50° C. to about +50° C.

Microbes or microorganisms that result in pathogenic infection of a host are well known. Thus, the methods and compositions of the invention can be used in the treatment or prophylaxis of infection by pathogenic microbes associated with any condition permitting delivery of the compositions of the invention to the site of infection, including, without limitation, the treatment of superficial or surgical wounds, burns or other significant epidermal damage (e.g., toxic epidermal necrolysis), urinary tract infections (e.g., cystitis and urethritis), vaginitis (e.g., vulvovaginitis and cervicitis), gingivitis, otitis externa, acne, external fungal infections, upper respiratory tract infections, gastrointestinal tract infections, subacute bacterial endocarditis and other bacterial or fungal infections to which the compositions of the invention can be effectively delivered. Pathogenic microbes which can be selectively killed in the practice of the invention include, without limitation, Streptococcus pyogenes, Streptococcus agalactiae, Staphylococcus aureus, S. pneumoniae, E. faecalis. S. epidermidis, Pseudomonas aeurginosa, Escherichia coli, Bacillus subtilis and other coliform bacteria, Candida albicans and T. rubrum, and other infectious bacteria and fungi.

The antiseptic compositions can be administered in any effective pharmaceutically acceptable form to warm-blooded animals, including humans and animal subjects, e.g., in topical dosage forms, such as a topical, buccal, or nasal spray or in any other manner effective to deliver a composition of the invention to a site of microbe infection. The route of administration will preferably be designed to obtain direct contact of the antiseptic compositions with the infecting microbes.

The present invention also contemplates that certain compositions described herein may be employed in the food processing and preparation industries to prevent and eliminate contamination of food with food borne bacteria, fungi and toxins. Thus, such compositions may be employed to reduce or inhibit microbial growth or otherwise abrogate the deleterious effects of microbial contamination of food. For these applications, the present compositions are applied in food industry acceptable forms such as additives, preservatives or seasonings.

For such applications, acceptable carriers may take the form of liquids, creams, foams, or gels and may additionally comprise solvents, emulsifiers, gelling agents, moisturizers, stabilizers, wetting agents, preservatives, sequestering agents, dyes, perfumes and other components commonly employed in food processing industry.

In specific embodiments, the contacting is performed for a time sufficient to kill the pathogenic agent or to inhibit the growth of the agent. In other embodiments, the present invention provides a method of decontaminating an environmental surface, area or atmosphere harboring harmful or undesired pathogens. In one such embodiment, the pathogenic agent is associated with an environmental surface and the method comprises contacting the environmental surface with an amount of the composition sufficient for decontaminating the surface. While it may be so desired, decontamination need not result in total elimination of the pathogen. In some embodiments, the compositions and methods may further comprise dyes, paints, and other marking and identification compounds so as to ensure that a treated surface has been sufficiently treated with the compositions of the present invention.

When the present compositions are administered as topical pharmaceuticals, it is contemplated that the compositions further comprise pharmaceutically acceptable adjutants, excipients, stabilizers, diluents, and the like. In still further embodiments, the present invention contemplates compositions further comprising additional pharmaceutically acceptable bioactive molecules. In the case of pharmaceutical activity, the effective amount relates to the dosage useful in achieving the desired end result. Such dosages are dependent upon the subject, i.e., age, size, etc. and can be easily ascertained by those skilled in this art.

Elimination of pathogenic microorganisms on various surfaces, especially hard surfaces where such organisms may stay active for relatively long periods of time, has long been a goal of those charged with cleaning and maintaining an antiseptic kitchen and bathroom in the home and in commercial and institutional settings such as hospitals, medical clinics, hotels and restaurants. A further goal has been to prevent the formation of allergens caused by growth of mold and mildew on bathroom surfaces.

This invention further relates to cleaning, sanitizing, disinfecting and mold and mildew inhibiting compositions for non-porous, hard surfaces such as glass (e.g., mirrors and shower doors), glazed porcelain, metallic (e.g., chrome, stainless steel, and aluminum), ceramic tile, enamel, fiberglass, Formica®, Corian® and plastic.

In general, the present invention contemplates compositions and methods that find use for environmental decontamination and for treatment of casualties in both military and terrorist attack. The inactivation of a broad range of pathogens, including vegetative bacteria and enveloped viruses and bacterial spores, combined with low toxicity in experimental animals, makes the present compositions suitable for use as general decontamination agents before a specific pathogen is identified. Preferred compositions of the present invention can be rapidly produced in large quantities and are stable for many months at a broad range of temperatures. These properties provide a flexibility that is useful for a broad range of decontamination applications.

For example, formulations of the present invention are effective at destroying many of the bacterial spores and agents used in biological warfare. In this regard, the compositions and methods of the present invention are useful in decontaminating personnel and materials contaminated by biological warfare agents. Solutions of the present compositions may be sprayed directly onto contaminated materials or personnel from ground based or aerial spaying systems. In certain of these applications, the present invention contemplates that an effective amount of the composition be contacted with contaminated materials or personnel such that decontamination occurs. Alternatively, personal decontamination kits can be supplied to military or civilians likely to become contaminated with biological agents.

The inactivation of a broad range of pathogens, including vegetative bacteria and enveloped viruses, combined with low toxicity, makes the present compositions particularly well-suited for use as general decontamination agents before a specific pathogen is identified.

Thus, certain embodiments of the present invention specifically contemplate the use of the present compositions in disinfectants and detergents to decontaminate soil, machinery, vehicles and other equipment, and waterways that may have been subject to an undesired pathogen. Such decontamination procedures may involve simple application of the formulation in the form of a liquid spray or may require a more rigorous regimen. Also, the present compositions can be used to treat crops for various plant viruses in place of or for use in combination with conventional antibiotics. The instant compositions may also be used to decontaminate farm animals, animal pens, surrounding surfaces, and animal carcasses to eliminate, for example, nonenveloped virus of hoof and mouth disease.

In addition to their use in decontamination of land and equipment, the formulations also find use as household detergents for general disinfectant purposes. Moreover, some embodiments of the present invention can be used to prevent contamination of food with bacteria or fungi (e.g., non-toxic compositions). This can be done either in the food preparation process or by addition to food as an additive, disinfectant, or preservative.

The inventive compositions can be used on hard surfaces in liquid or aerosol form. Accordingly, the foregoing components are admixed with one or more suitable aqueous or non-aqueous carrier liquids. The choice of carrier is not critical. However, it should be safe, and it should be chemically compatible with the inventive compositions. In some embodiments, the carrier liquid may comprise solvents commonly used in hard surface cleaning compositions. Such solvents should be compatible with the inventive compositions and should be chemically stable at the pH of the present compositions. Solvents for use in hard surface cleaners are described, for example, in U.S. Pat. No. 5,108,660, herein incorporated by reference in its entirety.

The present invention further relates to decontaminating a sample by treating the sample with the instant antimicrobial compositions such that bacteria, viruses, fungi or spores on the surface are killed or inhibited. The surfaces contemplated may be solid surfaces, such as the surfaces in homes, industrial facilities or medical facilities, or the surfaces of medical devices. Additionally the surface may be the surface of an organism and can be an internal or external organism surface. The surface further can be the surface of a food product.

### III. Compositions of the Invention

The present invention comprises microbiocidal or sporicidal compositions comprising an ionogenic surfactant, a metal chelating complex, and a solvent. According to the invention, the metal chelating complex comprises a metal compound, containing a monodentate, bidentate or polydentate ligand that exhibits affinity towards the hydrogen ion together with the surfactant in the proportion of about 1 to about (7-9) to the solvent. The metal chelating complex and the ionogenic surfactant are active ingredients of the microbiocidal or sporicidal compositions of the present invention. The active ingredients have disinfecting property against select microorganisms.

The chelating metal complex compound containing the ligand of this invention is a chelating complex compound with a metal such as copper, zinc, mercury, chromium, manganese, nickel, cadmium, arsenic, cobalt, aluminum, lead, selenium, platinum, gold, titanium, tin or combinations thereof. In one embodiment, the metal is a metal oxide, e.g., zinc oxide, or a metal salt.

The bi- and polydentate ligands are, for example, anions of natural amino acids, iminodiacetic or nitriletriacetic acids as well as carbon-substituted (in the α-position to the carboxylic group) derivatives of iminodiacetic and nitriletriacetic acids with various residues of amino acids fragments containing no aminocarboxylic group, alkylenediaminopolyacetic acid, as well as carbon-substituted (in the α-position to the carboxylic group) derivatives of polyalkylenepolyaminopolyacetic acids with various residues of aminoacetic fragments containing no aminocarboxylic group, derivatives of ω-phosphoncarboxylic and ethylenediphosphontetrapropionic acids, derivatives of ethylenetetra(thioacetic) and diethylenetrithiodiacetic acids, monoamine complexones, in which carboxylic groups are replaced by phosphonic groups, or mixtures thereof.

The chelating metal complex compound containing a monodentate, bidendate or polydentate ligand can be a chelating complex compound with at least one amino acid such as for example isoleucine, phenylalanine, leucine, lysine, methionine, threonine, tryptophan, valine, alanine, glycine, arginine, histidine, or mixtures thereof.

An embodiment of the invention comprises a microbiocidal or sporicidal composition containing an ionogenic surfactant, a metal chelating complex and a solvent, wherein the chelating complex comprises a chelating metal complex compound containing a monodentate, bidentate or polydentate, ligand that exhibits affinity to hydrogen ion, and the solvent comprises a mixture of water and an aliphatic alcohol (C₁-C₈) with the following ratio, weight %:

| | |
|---|---|
| Chelating complex metal compound, containing | about 1-30 |
| a monodentate, bidentate or polydendate ligand | |
| that exhibits affinity to hydrogen ion | |
| Ionogenic surfactant | about 0.1-15 |
| Aliphatic alcohol (C₁-C₈) | about 0.5-95 |
| Distilled water | remainder |

Exemplary chelating metal complex compounds comprise glycinatecopper chloride complex and an ethylenediaminotetraacetate zinc complex.

Suitable halogen containing ionogenic compounds may be selected, for example, from compounds comprising chloride, fluoride, bromide and iodide ions. In preferred embodiments, suitable cationic halogen containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyldimethylethylammonium bromide, cetyltributylphosphonium bromide, dodecyltrimethylammonium bromide, and tetrad cetyltrimethylammonium bromide. In particularly preferred embodiments, the cationic halogen containing compound is CPC, although the compositions of the present invention are not limited to formulation with a particular cationic containing compound.

Exemplary ionogenic surfactants comprise cetylpyridinium halogenides and cetyltrimethylammonium halogenides.

Metal complex compounds are useful disinfecting and antibacterial preparations. They are bactericidal reagents exhibiting a broad range of antibacterial action, irreversibly killing pathogenic microorganisms. The mechanism of action of metal complex compounds is based on blocking amino acid groups of a protein shell and enzyme systems of microorganisms. At the first stage, there are formed associates with a chelating complex and then a monodentate, bidentate or polydentate ligand is substituted by an amino acid group of a protein, which leads to a complete blocking of metabolic processes in microorganisms and subsequently to their death.

The proposed compounds relate to the IY toxicity class. Doses of the microbiocidal or sporicidal composition of the present invention do not cause a pronounced toxic or irritating effect on skin or mucosa.

The proposed compositions based on chelating metal complex compounds do not exert influence on animal or human organisms because the compounds containing amino acid groupings are withdrawn from the organism by the exchange reaction. Bactericidal chelating complexes do not affect the most important living functions of the organism.

The proposed bactericides relate to metal complexes with chelating ligands, which are obtained in the alkaline and not in the acidic pH range. Therefore, the proposed compositions compared to the analogs have a broader field of application because they are ecologically safe and possess low toxic and hygienic characteristics based on a different mechanism of bactericide action. In addition, the proposed compositions exhibit an increased chemical stability towards environmental impact (i.e., stability constants of the proposed complexes are several orders higher than those of the closest analogs).

Useful monodentate, bidentate or polydentate ligands include ligands exhibiting affinity towards hydrogen ion, which determines their ability to be substituted by an amino group of protein in a microorganism.

A molecule of the proposed bactericide contains a metal ion, preferably, for example, copper (II) and zinc as well as monodentate, bidentate or polydentate ligands, exhibiting affinity towards hydrogen ion, such as ammonia, mono-, di- and triethanolamines and others.

The pH of the obtained bactericidal compositions is about ≧7.0.

For the synthesis of bactericides, use is made of metal salts. The synthesis is carried out in aqueous solutions by stirring the ingredients at room temperature. The monodentate ligands used are water soluble substances that display affinity towards a hydrogen ion.

The distinguishing characteristic of the present bactericide compositions is that the interaction (mixing) of the ingredients takes place in neutral and alkali media at pH ≧about 7.0 in the absence of mineral acids.

As for the parameters of the disinfecting activity, it is established that the present microbiocidal and sporicidal compositions are sufficient and do not require the use of any additional disinfecting preparations, for example, Chlorohexydine, hydrogen peroxide, etc.

The method for synthesis of the glycinatecopper chloride complex and ethylenediaminotetraacetate zinc complex is known from the following sources: Ley, Berichte, V. 42, S. 371; Hofmeister, “Beittage zur Kenntiniss der Amidosäurcn” Annalen der Chemie, 1877 V. 189, S.36; “Synthetic Production and Utilization of Amino Acids”, Ed. T. Kaneko, Y. Izumi, I. Chibata, Wiley, N.Y., 1974; and Dyatlova N. M. et al., Complexones and Metal Complexonates, M.:—<<Khimiya>> 1988).

The antimicrobial activity of the glycinatecopper chloride complex, ethylenediaminotetraacetate zinc complex and compositions thereof was investigated in the Scientific Research Disinfectology Institute, Moscow.

The ingredients ratio in the proposed compositions is selected so as to provide for optimal technological characteristics of the preparation and for retaining the desired stability.

The concentrations ranges in the compositions:

| | |
|---|---|
| Chelating metal complex | about 1%-30% |
| Ionogenic Surfactant (e.g., quaternary ammonium | about 0.1%-15% |
| halogenides-, C₁₂-C₁₆-alkyltrimethylammonium, | |
| di(C₈-C₁₀-alkyl)dimethylammonium, in particular | |
| cetylpyridinium and cetyltrimethylammonium | |
| halogenides) | |
| Aliphatic alcohol (C₁-C₈) | about 0.5%-95% |
| Water or other components | about 3%-98% |

The proposed concentration ranges for the ingredients in the composition are determined by the objective to achieve the above mentioned bactericidal, fungicidal and sporocidal activity. The technical result is possible to achieve by making use of, as ionogenic surfactants, quaternary ammonium halogenides, in particular C₁₂-C₁₆ alkyltrimethylammonium, di(C₈-C₁₀-alkyl)dimethylammonium, C₁₂-C₁₆-alkylpyridinium, in particular cetylpyridinium and cetyltrimethylammonium halogenides.

### VI. Formulation and Delivery of the Microbiocidal/Sporicidal Compositions

A. Pharmaceutical Compositions

The microbiocidal or sporicidal compositions of the present invention can be incorporated into pharmaceutical compositions with a pharmaceutically acceptable carrier suitable for administration. As used herein, “pharmaceutically acceptable carrier” is intended to include any and all solvents, dispersion media coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline Ringer's solutions, and dextrose solution. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with a transdermal (i.e., topical), route of administration, transmucosal (e.g., tunica mucosa vaginae), and rectal administration. Solutions or suspensions used for transdermal, transmucosal, or rectal administration can include the following components: a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. In all cases, the composition must be sterile and should be fluid to the extent that the need for easy topical application exists. The proper fluidity can be maintained, for example, by the use of a coating such as lecithin, by the maintenance of the required particle size in the case of dispersion and by the use of surfactants. The microbiocidal or sporicidal composition must be stable under the conditions of manufacture and storage. The carrier can be a solvent or dispersion medium containing, for example, water, ethanol, polyol (for example, glycerol, propylene glycol, and liquid polyethylene glycol, and the like), and suitable mixtures thereof.

When the present compositions are administered as topical pharmaceuticals, it is contemplated that the compositions further comprise pharmaceutically acceptable adjutants, excipients, stabilizers, diluents, and the like. In still further embodiments, the present invention contemplates compositions further comprising additional pharmaceutically acceptable bioactive molecules. In the case of pharmaceutical activity, the effective amount relates to the dosage useful in achieving the desired end result. Such dosages are dependent upon the subject, i.e., age, size, etc. and can be easily ascertained by those skilled in this art.

For topical applications, the pharmaceutically acceptable carrier may take the form of liquids, creams, lotions, or gels and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration. Compositions of the invention may be impregnated into absorptive materials, such as sutures, bandages, and gauze, or coated on to the surface of solid phase materials, such as staples, zippers and catheters to deliver the compositions to a site of microbe infection. Other delivery systems of this type will be readily apparent to those skilled in the art.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration.

In many cases, it will be preferable to include isotonic compounds, for example, sugars, polyalcohols such as manitol, sorbitol, and sodium chloride in the composition. Prolonged absorption of the compositions can be brought about by including in the composition a compound which delays absorption, for example, aluminum monostearate and gelatin.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art. The microbiocidal or sporicidal composition can contain any of the following ingredients, or compounds of a similar nature: a lubricant such as magnesium stearate or Sterotes and/or a glidant such as colloidal silicon dioxide. Further, the composition of the present invention can be used in combination with other chemical elements including hydrophilic compounds and hydrophobic polymers, as specifically set forth in U.S. Pat. No. 5,417,968, to provide such products as a prophylactic skin barrier providing antiviral, antibacterial and antifungal protection. Furthermore, the microbiocidal or sporicidal composition can be combined with various antibacterial and antifungal compounds, for example, parabens, chlorobutanol, phenol, ascorbic acid, thimerosal, and the like.

The compounds can also be prepared as pharmaceutical compositions in the form of suppositories (e.g., with conventional suppository bases such as cocoa butter and other glycerides) or retention enemas for rectal delivery. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Douche preparations or solutions for vaginal irrigation may be made by combining the active ingredients with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants and preservatives.

Preparations used to treat dandruff may be made by combining the active ingredients with a pharmaceutically acceptable carrier. The active ingredients can be used alone or in combination with other common topical preparations successfully used to treat dandruff, e.g., ketoconazole, zinc pyrithione, selenium sulfide, sulfur and coal tar. The preparations used to treat dandruff are generally shampoos, formulations well-known in the art. Zinc pyrithione decreases the turnover of the rapidly dividing epidermal cells. Coal tar has antiseptic, antipruitic (anti-itching), and exfoliating properties.

B. General Formulation

As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, ocular and rectal administration. Solutions or suspensions used for parenteral, intradermal, or subcutaneous application can include the following components: a sterile diluent, such as water, for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or a dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol and sodium chloride in the composition. Prolonged absorption of the injectable compositions can be brought about by including in the composition a compound which delays absorption, for example, aluminum monostearate and gelatin.

Sterile injectable solutions can be prepared by incorporating the microbiocidal or sporicidal compositions in the required amount in an appropriate solvent with one or a combination of ingredients enumerated above, as required, followed by filtered sterilization. Generally, dispersions are prepared by incorporating the microbiocidal or sporicidal compositions into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient, plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes: a glidant such as colloidal silicon dioxide; a sweetening compound such as sucrose or saccharin; or a flavoring compound such as peppermint. methyl salicylate, or orange flavoring.

For administration by inhalation, the compounds are delivered in the form of an aerosol spray from a pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic add derivatives. Transmucosal administration can be accomplished through the use of nasal sprays or suppositories. For transdermal administration, the active compounds are formulated into ointments, salves, gels, or creams, as generally known in the art.

The microbiocidal and sporicidal compositions can also be prepared as pharmaceutical compositions in the form of suppositories (e.g., with conventional suppository bases such as cocoa butter and other glycerides) or retention enemas for rectal delivery.

In one embodiment, the microbiocidal or sporicidal compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In another embodiment, the microbiocidal or sporicidal compositions of the present invention are combined with, or co-administered with, one or more of the following formulations: a preoperative skin wash (e.g., Trizenol, Triseptin, and/or Actiprep), a topical antifungal preparation (e.g., Mitrazol), a wound cleanser (such as Allclenz), a topical anti-infective (e.g., Panafil and/or Lodosorb), an antibiotic-based topical anti-acne preparation (e.g., Akno-mycin); a dermatitis face wash (such as Ovace), a wound debrider (such as disclosed in U.S. Pat. No. 6,548,556 and in U.S. Patent Application Nos. 20030198631 and 20030198632); a skin protectant (such as disclosed in U.S. Pat. Nos. 5,482,714 and 5,558,872); other antimicrobial compositions (such as disclosed in U.S. Patent Application No. 20020022660); and numerous shampoos and skin scrubs.

In another embodiment, the microbiocidal or sporicidal compositions of the present invention are combined with, or co-applied with, one or more of the following formulations to clean and disinfect medical devices and surfaces: a chemical sterilant (such as the disinfectant solution disclosed in U.S. Pat. Nos. 5,327,542 and 6,005,348); other bactericidal, fungicidal, tuberculocidal, and viricidal disinfectant/sterilant formulations (such as the disclosed in U.S. Pat. No. 5,863,547 and in U.S. Patent Application No. 20030157192).

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, as well as the limitations inherent in the art of preparing such an active compound for the treatment of individuals.

C. Formulations of the Microbiocidal/Sporicidal Composition for Other Delivery Modalities and Applications

The composition of the present invention is useful alone as an effective antiviral, antibacterial and antifungal substance to provide numerous products including, but not limited to, ointments, disinfectant hand soaps, hypo-allergenic hand care creme, shampoo, face soap, douche, laundry products, dish washing products (including a bar glass dip), bathroom cleaning products, dental products (e.g., mouthwash, dental adhesive, saliva injector filters, water filtration), first-aid ointments and sprays, hand washes, foot washes, eye ointments or washes, treatment for toenail fungus, topical treatments for superficial infections of the skin, i.e., a drug, preoperatice skin wash or wound wash, as well as a device disinfectant and deodorizing products. As such, it is contemplated that this composition can be delivered in a number of different modalities, including liquid, spray paste, gel, powders, dehydrated tablet, or incorporated into liquid, solid or dry soaps, cleansers and cleaners. The preparation of pastes, gels, powders and concentrated dehydrated tablets taught by the present invention are readily known by those skilled in the art. It is also contemplated that a paste, gel or solid modality may be preapplied to a wipe, gauze or adhesive bandages in effective quantities for ease and convenience of packaging, storage, portability and dispensing. For example, a small skin lesion associated with HIV can be effectively treated with a localized application. A localized application can be achieved using an adhesive bandage delivery modality. Moreover, the present compositions can be sprayed into an atmosphere to inactivate harmful microorganisms in the atmosphere. Such spray disinfectants are readily formulated by the skilled artisan and the choice of carrier is within the skill in the art.

The composition can be used in aerosolized, misted, vaporized, fogged, humidified or other forms used to produce micronized particles of the composition that can remain in suspension in the air for long periods of time. The micronized particles act much like a fumigant to provide total coverage to all sides of a surface that may be infected with pathogens. In any of these forms, the composition is able to intercept fungi bacteria, and viruses, spores, and/or resting (dormant) stages of the pathogen in the air. The composition prompts the pathogen to vegetate and/or otherwise vitalize the dormant stage of the pathogen and the formulation as defined by itself or in combinations with other components of a formulation capable of killing the vegetative stage and/or spores and/or resting spores and/or resting stage of the pathogen by contact and/or action of the total formulation on the pathogen.

D. Combination Therapies

The antimicrobial compounds described herein are useful in treating infections and physiological responses to infection, for example, inflammation, tissue necrosis, septicemia and other disorders resulting from infection as described. In a preferred aspect, the antimicrobial compounds are formulated into pharmaceutical compositions for dermatological therapeutics or prophylactics. In addition, where an antimicrobial is provided through oral dosing, for example Lamisil, the present antimicrobial compounds can be applied locally to enhance oral therapy. Dosages of the antimicrobial compounds suitable for topical formulations are provided above. The following are non-limiting examples of systemic and topical dermatological preparations, each capable of being used in conjunction with the antimicrobial compositions, or otherwise reformulated to contain the antimicrobial composition described herein.

Zimycan was developed for Candida-associated diaper dermatitis in infants. This topical miconazole-based product in a zinc oxide and petrolatum base will compete against steroid-based prescription treatments. The present antimicrobial compounds can be formulated into topical preparations having Zimycan for additional antifungal and broad spectrum antimicrobial activity.

Seboride is a topical gel that combines the long-lasting effect of the antifungal agent, ketoconazole, with the fast-acting, mid-potency steroid, desonide. The formulation provides higher antifungal efficacy with convenient once-a-day dosing for only two weeks. Seboride is targeted for seborrheic dermatitis, a disease that affects annually between three to five percent of the US adolescent and adult population. The present antimicrobial compounds can be formulated into topical preparations having Seboride for additional antifungal and broad spectrum antimicrobial activity.

Sporamelt is an enhanced version of the oral antifungal itraconazole. Sporamelt features a novel delivery technology that allows for once-daily oral dosing in skin and nail mycoses and vaginal candidosis. Sporamelt is intended to allow once-daily dosing for pulse treatment in fungal infections. This product is a systemic oral formulation, but topical preparations of the present antimicrobial compounds complement oral Sporamelt therapy, providing additional antifungal and additional broad spectrum antimicrobial activity.

Liarozole and Rambazole are members of a class of molecules called RAMBAs (Retinoic Acid Metabolism Blocking Agents). RAMBAs have been shown to be safer and less irritating than retinoids such as Accutane, Retin-A, and Soriatane. Oral treatment with Liarozole and Rambazole have demonstrated positive effects in providing therapeutic effect against diseases such as ichthyosis (congenital forms as well), psoriasis, and acne. Rambazole and Liarozole use the body's own retinoic acid stores, are the first dermatological products based on this pharmacological mechanism of action, and significantly reduce the long term toxic side effects that commonly occur with conventional oral and topical refined derivatives. Rambazole® has demonstrated a better therapeutic index than Liarozole in oral studies. Topical treatment has yielded impressive results and an even safer therapeutic index than observed with oral treatment. When these products are used in oral formulations for systemic therapy, topical preparations of the present antimicrobial compounds complement oral Liarozole and Rambazole therapy. The present antimicrobial compounds can also be formulated into topical preparations having Liarozole and Rambazole, providing additional antifungal and broad spectrum antimicrobial activity therapy.

Azoline is a novel triazole derivative that has shown to be 5 times more active than itraconazole in dermatophyte infections in animals. It combines this superior efficacy with a 5 to 10 times lower interaction potential with drug metabolizing enzymes in the liver as compared to earlier azole derivatives. This product is a systemic oral formulation, but topical preparations of the present antimicrobial compounds complement oral Azoline therapy, providing additional antifungal and broad spectrum antimicrobial activity.

Hivenyl is a highly selective antihistamine blocker that does not penetrate the blood brain barrier and, as such, eliminates the risk for any kind of sedation. One week testing in volunteers (up to 15 times the required daily dose) proved to be safe and effective in inhibiting a histamine induced wheal and flare reaction. Hivenyl has been extensively tested for possible secondary cardiovascular effects, and no negative effects have been seen. Hivenyl treats dermatological allergies. This product is a systemic oral formulation, but topical preparations of the present antimicrobial compounds complement oral Hivenyl therapy, providing localized broad spectrum antimicrobial activity, killing the organisms that produce the inflammation and urticadia seen in dermatological allergies.

Atopik is being evaluated as a potent topical treatment for eczema/dermatitis. It is a phosphodiesterase 4 (PDE₄) inhibitor that has been specially formulated in a topical form to avoid the side effects associated with systemic inhibition of PDE₄. The early first human results support equivalent potency between Atopik and betamethasone valerate, a potent steroid, in suppressing contact and irritant dermatitis. Topical preparations of the present antimicrobial compounds complement therapy with phosphodiesterase 4 inhibitors, providing localized broad spectrum antimicrobial activity, killing the organisms that produce the inflammation and urticadia seen in contact and irritant dermatitis.

Ketanserin is a serotonin II antagonist used as a topical agent in the treatment of chronic wounds, especially those of diabetic and arterial origin. The drug acts through stimulation of granulation tissue, resulting in faster wound closure. Topical preparations of the present antimicrobial compounds complement therapy with serotonin II antagonists, providing localized broad spectrum antimicrobial activity and permitting antiseptic conditions for optimum wound healing.

Oxatomide is a topically active broad-spectrum, anti-allergy compound shown to topically suppress itching in atopic eczema, pain and inflammation in burns (UV, chemical and thermal), as well as various skin conditions associated with itching. Topical preparations of the present antimicrobial compounds complement therapy with Oxatomide, providing additional localized broad spectrum antimicrobial activity, particularly against eukaryotic microbes, reducing inflammation, and permitting antiseptic conditions for optimum healing.

Ecalcidene is an oral vitamin D₃ derivative, having immunological effects at doses that are 10 to 100 times lower than the doses causing toxicity associated with hypercalcemia. The drug is useful in treating psoriasis, osteoporosis, organ transplant rejection, and chronic inflammatory disorders, (e.g., rheumatoid arthritis). This product is a systemic oral formulation, but topical preparations of the present antimicrobial compounds complement oral Ecalcidene therapy, providing additional antifungal and broad spectrum antimicrobial activity.

### VII. Treatment of Disease and Disorders

A. Prophylactic and Therapeutic Uses of the Compositions of the Invention

The microbiocidal or sporicidal compositions of the present invention are useful in potential prophylactic and therapeutic applications implicated in a variety of disorders in a subject (See Diseases and Disorders). Diseases and disorders that are characterized by increased (relative to a subject not suffering from the disease or disorder) levels of biological activity of bacteria, yeast, fungi, or viruses can be treated with AMC-based therapeutic compounds that antagonize (i.e., reduce or inhibit) the growth and that can be administered in a therapeutic or prophylactic manner. Levels of bacteria, yeast, fungi, or viruses can be readily detected by obtaining a patient tissue sample (e.g., from biopsy tissue or scraping) and assaying it in vitro for bacteria, yeast, fungus, or virus levels by appropriate culture followed by cytochemical staining and/or inspection using microbiological techniques well known in the art, e.g., Gram staining. Alternatively, samples may be assessed for the presence of microbial, fungal or viral nucleic acids, using molecular biological techniques well-known in the art, e.g., polymerase chain reaction. Other methods useful in the measurement of bacteria, yeast, fungus, or virus levels well-known within the art include, but are not limited to, immunoassays (e.g., by Western blot analysis, immunoprecipitation followed by sodium dodecyl sulfate (SDS) polyacrylamide gel electrophoresis. immunochemistry, etc) and/or hybridization assays to detect expression of mRNAs (e.g., Northern assays. dot blots, in situ hybridization, and the like).
i. Prophylactic Methods

In one aspect the invention provides a method for preventing a disease or condition associated with a microorganism in a subject by administering to the subject a microbiocidal or sporicidal composition. Subjects at risk for a disease that is caused by or contributed to by bacterial, yeast, fungi, or virus proliferation can be identified by, for example, any or a combination of diagnostic or prognostic assays, as described herein. Administration of a prophylactic microbiocidal or sporicidal composition of the present invention can occur prior to the manifestation of symptoms characteristic of the aberrancy, such that a disease or disorder is prevented or, alternatively, delayed in its progression. Depending upon the type of aberrancy, for example, a microbiocidal or sporicidal composition of the present invention that acts as an antagonist to bacterial, yeast, fungal or viral proliferation can be determined based on screening assays described herein.
ii. Therapeutic Methods

Another aspect of the invention includes methods of inhibiting bacterial, yeast, fungal, or viral activation and/or proliferation in a subject for therapeutic purposes. The modulatory method of the invention involves contacting a cell with a compound of the present invention that inhibits bacterial, yeast, fungal, or viral activation and/or proliferation. These methods can be performed in vitro (e.g., by culturing the cell with the microbiocidal or sporicidal composition) or, alternatively, in vivo by administering the microbiocidal or sporicidal composition to a subject, e.g., applying a microbiocidal or sporicidal composition topically). As such, the invention provides methods of treating an individual afflicted with a disease or disorder manifested by aberrant activation or proliferation of bacteria, yeast, fungi or viruses.

B. Determination of the Biological Effect of Microbiocidal/Sporicidal Compositions

In various embodiments of the invention, suitable in vitro or in vivo assays are performed to determine the effect of a specific microbiocidal or sporicidal composition and whether its administration is indicated for treatment of the affected tissue in a subject. Compounds for use in therapy can be tested in suitable animal model systems including, but not limited to rats, mice, chicken, cows, monkeys, rabbits, and the like, prior to testing in human subjects. Similarly, for in vivo testing, any of the animal model systems known in the art can be used prior to administration to human subjects. In various specific embodiments, in vitro assays can be performed with representative cells of the type(s) involved in the patient's disorder to determine if a given AMC-based composition exerts the desired effect upon the particular cell type(s).

C. Diseases and Disorders

Microorganism activation and/or proliferation is associated with numerous diseases, all of which could be affected by administration of a microbiocidal or sporicidal composition. The invention provides for both prophylactic and therapeutic methods of treating a subject at risk of or susceptible to a disorder or having a disorder associated with aberrant microorganism activations and/or proliferation, e.g., but not limited to, bacterial, yeast, fungal, or viral activation and/or proliferation.
i. Use of Microbiocidal/Sporicidal Compositions to Prevent or Treat Bacterial Infections

The microbiocidal or sporicidal compositions of the present invention are useful in the prevention or therapeutic treatment of Pseudomonas infection, when used in an effective amount. One Pseudomonas species is an important pathogen of humans. Pseudomonas aeruginosa is an opportunistic pathogen that is a leading cause of hospital-acquired (nosocomial) infections.

The microbiocidal or sporicidal compositions of the present invention are useful in the prevention or therapeutic treatment of Enteric bacterial infection, when used in an effective amount. Enteric bacteria are Gram-negative rods with facultative anaerobic metabolism that live in the intestinal tracts of animals. This group consists of E. coli and its relatives, the members of the family Enterobacteriaceae. A few strains of E. coli are pathogenic, e.g., E. coli strain 0157:H7. Pathogenic E. coli causes intestinal tract infections and urinary tract infections.

The microbiocidal or sporicidal compositions of the present invention are useful in the prevention or therapeutic treatment of cocci bacterial infection, when used in an effective amount. The pyogenic cocci are spherical bacteria that cause various suppurative (pus-producing) infections in animals. Included are the Gram-positive cocci S. aureus, S. pyogenes and S. pneumoniae, and the Gram-negative cocci Neisseria gonorrhoeae and N. meningitidis. These bacteria are leading pathogens of humans. It is estimated that they produce at least a third of all the bacterial infections of humans, including strep throat, pneumonia, food poisoning, various skin diseases and severe types of septic shock, gonorrhea and meningitis.

Further, two species of Staphylococcus live in association with humans: S. epidermidis, which lives normally on the skin and mucous membranes, and S. aureus, which may occur normally at various locales but in particular on the nasal membranes (nares). S. epidermidis is sometimes a pathogen. S. aureus always has the potential to cause disease and so is considered a pathogen. S. aureus can produce a wide range of infections, and it often occurs as normal flora of humans (on skin, nasal membranes arid the GI tract), which ensures that it is readily transmitted from one individual to another. Different strains of S. aureus differ in the range of diseases they can cause, including boils and pimples, wound infections, pneumonia, osteomyelitis, septicemia, food intoxication, and toxic shock syndrome. S. aureus is the leading cause of nosocomial (hospital-acquired) infections by Gram-positive bacteria. Also, it is notoriously resistant to penicillin and many other antibiotics.

S. pyogenes, more specifically the Beta-hemolytic Group A Streptococci, like S. aureus, causes an array of suppurative diseases and toxinoses (i.e., diseases due to the production of a bacterial toxin), in addition to some autoimmune or allergic diseases. S. pyogenes is rarely found as normal flora (<1%), but it is the main streptococcal pathogen for man, most often causing tonsillitis or strep throat. Streptococci also invade the skin to cause localized infections and lesions, and produce toxins that cause scarlet fever and toxic shock.

Streptococcus pneumoniae is the most frequent cause of bacterial pneumonia in humans. It is also a frequent cause of otitis media (infection of the middle ear) and meningitis. The bacterium colonizes the nasopharynx and from there gains access to the lung or to the eustachian tube. If the bacteria descend into the lung they can impede engulfment by alveolar macrophages if they possess a capsule that somehow prevents the engulfment process. Thus, encapsulated strains are able to invade the lung and are virulent (cause disease) and noncapsulated strains, which are readily removed by phagocytes, are nonvirulent.

The microbiocidal or sporicidal compositions of the present invention are useful in the prevention or therapeutic treatment of Bacillus bacterial infection, when used at a concentration of 0.1-100%. At least 48 species, including B. subtilis, are known, but only B. anthracis and B. cereus cause disease in humans. B. anthracis is responsible for the disease anthrax. This is a disease primarily of animals, but humans can acquire anthrax via handling, inhaling or ingesting contaminated animal products.

Anthrax infections are classified by route of entry. In cutaneous anthrax, Bacillus spores enter the skin through a cut or animal bite and germinate. A small red lesion develops after 1-7 days, eventually producing local necrosis (i.e., the “black eschar”). Spread of the bacteria causes regional lymph tenderness, which may be followed by toxic septicemia and death. Only about 5% of cutaneous infections become septic inhalation anthrax. In inhalation anthrax, Bacillus spores are inhaled and ingested by aveolar macrophages. These carry the bacteria to the regional lymph nodes, causing necrotic hemorrhaging that leads to death.

B. cereus is predominantly responsible for food poisoning in humans. B. cereus food poisoning results from the ingestion of preformed enterotoxins, producing predominantly vomiting and diarrhea. The vomiting form is most often associated with ingestion of a heat stable toxin from contaminated rice, while the diarrheal form is most often associated with ingestion of a heat labile toxin from contaminated meat or vegetables.

The microbiocidal or sporicidal compositions of the present invention are useful in the prevention or therapeutic treatment of infection leading to acne, when used in an effective amount. The bacteria in acne include Propionibacterium acnes (P. acnes), Propionibacterium granulosum, and Staphylococcus epidermidis. The numbers of the yeast Malassezia furfur also increase in certain types of acne, including, e.g., non-inflammatory acne, inflammatory acne, and acne congloblate.

P. acnes can produce active enzymes and inflammatory mediators that may contribute to the activity of acne. These include: lipases, proteases, hyaluronate lyase, phosphatases, and smooth-muscle contracting substances.

Though all pimples start the same way they can take many forms and may react differently for different people. All acne begins with one basic lesion, the comedo, an enlarged hair follicle plugged with oil and bacteria. Invisible to the naked eye, the comedo lurks beneath the surface of the skin waiting for the right conditions to grow into an inflamed lesion. As the skin continues to produce more oil, bacteria flourishes within the swollen follicle. The surrounding skin becomes increasingly inflamed as your white blood cells fight against the intruders.
ii. Use of Microbiocidal/Sporicidal Compositions to Prevent or Treat Fungal/Yeast Infection

The microbiocidal and sporicidal compositions of the present invention are useful in the prevention or therapeutic treatment of dermatophyte infection, e.g., anthropophilic, zoophilic or geophilic, when used in an effective amount. Dermatophytes are fungi that can cause infections of the skin, hair, and nails due to their ability to utilize keratin. The organisms colonize the keratin tissues and inflammation is caused by host response to metabolic by-products. These infections are known as ringworm or tinea in association with the infected body part.

Occasionally the organisms do invade the subcutaneous tissues, resulting in kerion development. The organisms are transmitted by either direct contact with an infected host (human or animal) or by direct or indirect contact with infected, exfoliated skin or hair in combs, hair brushes, clothing furniture, theatre seats, caps, bed linens, towels, hotel rugs, and locker room floors. Depending on the species, the organism may be viable in the environment for up to 15 months. There is an increased susceptibility to infection when there is a preexisting injury to the skin such as scars, burns, marching, excessive temperature and humidity.

Dermatophytes are classified as anthropophilic, zoophilic or geophilic according to their normal habitat. Anthropophilic dermatophytes are restricted to human hosts and produce a mild, chronic inflammation. Zoophilic organisms are found primarily in animals and cause marked inflammatory reactions in humans who have contact with infected cats, dogs, cattle, horses, birds, or other animals. This is followed by a rapid termination of the infection. Geophilic species are usually recovered from the soil but occasionally infect humans and animals. They cause a marked inflammatory reaction that limits the spread of the infection and may lead to a spontaneous cure but may also leave scars.

Anthropophilic, zoophilic and geophilic dermatophytes include, Epidermophyton floccosum; Microsporum audouinii; Microsporum ferrugineun; Trichophyton concentricum; Trichophyton kanei; Trichophyton megninii; Trichophyton mentagrophytes; Trichophyton raubitschekii; Trichophyton rubrum; Trichophyton schoenleinii; Trichophyton soudanense; Trichophyton tonsurans; Trichophyton violaceum; Trichophyton yaoundei; Microsporum canis (cats, dogs, etc), Microsporum equinum (horses), Microsporum nanum (pigs); Microsporum persicolor (rodents); Trichophyton equinum (horses); Trichophyton mentagrophytes (granular; rodents, rabbits, hedgehogs, etc); Trichophyton simii (monkeys); Trichophyton verrucosum (cattle); Microsporum gypseum; Trichophyton ajelloi; and Trichophyton terrestre. Infrequently isolated (less than 1%) are Epidermophyton floccosum, Microsporum audouinii, M. canis, M. equinum, M. nanum, M. persicolor, Trichophyton equinum, T. kanei, T. raubitschekii, and T. violaceum. The dermatophytoses include favus and infections due to species of Epidermophyton, Micro-sporum, and Trichophyton.

Specific conditions included under the term dermatophytoses include, but are not limited to, e.g., Beard ringworm, Kerion, Scalp ringworm, Mycotic sycosis; dermatophytic onychia, dermatophytosis of nail, onychomycosis, ringworm of nails, dermatophytosis of hand, hand ringworm, Athlete's foot, Dermatophytosis of foot, Foot ringworm, Ringworm of body, Tokelau, Dhobi itch, Groin ringworm, Jock itch, disseminated dermatophytosis, granulomatous dermatophytosis and dermatophytosis, unspecified, Ringworm, and NOS.

T. rubrum is the most frequently isolated anthropophilic dermatophyte. It is found on the feet, nails, body, groin, and sometimes the scalp. This fungus is the most common cause of jock itch. It also causes fungal infections of the toes and body. The microbiocidal compositions of the present invention are useful, therefore, in the prevention or treatment of jock itch and Athlete's foot.

Dandruff (pityriasis capitis) occurs when the scalp sheds larger than normal amounts of dead epidermal cells. It is sometimes associated with seborrhea where sebum production is excessive. Dandruff shares some features with seborrheic dermatitis, and both conditions are frequently treated with common topical medications. Seborrheic dermatitis generally affects body sites in addition to the scalp, including the forehead, nasolabial fold, eyelash and eyebrow regions, and the outer ear.

Dandruff appears on the scalp as small white or gray scales. In the presence of seborrhea, the scales may appear greasy and yellow in color. The greasy scales combine with exudates to form crusts, beneath which the scalp is red and moist. Shampooing removes the scales temporarily however they return within several days.

Dandruff is associated with fewer cell layers in the outer most portion of the epidermis, however the cells are often irregular and display a rapid turnover rate. For many years dandruff has been associated with the presence of yeast/fungi of the genus Malassezia or Pityrosporum. At this time, the species Pityrosporum ovale, is considered the main causative agent, although some investigators argue that the altered flora of the scalp is secondary to increased epidermal proliferation. Seborrheic dermatitis has also been associated with the activities of Pityrosporum fungi. Effective therapies of both dandruff and seborrheic dermatitis have been linked to agents that inhibit these organisms.

The microbiocidal or sporicidal compositions of the present invention are useful in the prevention or therapeutic treatment of yeast infection, when used in an effective amount. Candidiasis is an infection caused by Candida, a yeast-like fungus, e.g., Candida albicans; C. glabrata; C tropicalis; C. parapsilosis; and C. krusei. Candidiasis usually affects the skin and mucous membranes (e.g., soft, moist areas around body openings, like the mouth and anus). The illness can take several different forms, each with different symptoms. The specific form of candidiasis depends on many factors, including the child's age and general health.

In healthy newborns, the most common form of candidiasis is a diaper rash. Skin in the diaper area becomes red and tender, especially inside skin folds and creases. In general, any diaper rash that lasts for 3 days or longer may be candidiasis. Also in healthy newborns, candidiasis may appear as oral “thrush.” In oral thrush, the Candida fungus invades parts of the mouth and throat, causing cracks in the corners of the mouth and whitish or yellowish patches on the lips, tongue, palate, and inside the cheeks. When these patches are scraped or rubbed, pinpoint areas of bleeding can be seen underneath. Often, a baby with oral thrush may have no other symptoms than the patches. Sometimes the patches are painful, however, and the child has problems feeding or is generally fussy and irritable. Newborns can develop thrush from mothers who have vaginal “yeast infections” at the time of delivery. When this happens, symptoms of oral thrush usually begin 7 to 10 days after birth.

Children of any age may develop Candida paronychia, an infection of the skin around the nails. Fingernails are most often affected, especially in children who spend a lot of time with their hands in water. The cuticle and skin around the nails becomes swollen, red, and sometimes painful. The fingernails may grow to be abnormally shaped or colored, or may actually lift away from the skin.

Older girls and women may develop Candida vulvovaginitis, an infection of the vagina and the area around the vaginal opening. This is also commonly called a vaginal “yeast infection.” Symptoms include: vaginal pain, itching, or redness, a thick, white “cheesy” vaginal discharge, pain or discomfort in urination, and sometimes whitish or yellowish patches on the skin of the vaginal area (these loco similar to the patches seen in the mouth of a baby with oral thrush).

Also, in both sexes, any part of the body that is constantly moist, warm, and dark can be a site of Candida infection. This is especially true of skin folds in the areas of the scrotum, underarms, inner thighs, areas between fingers and toes, and the skin over the base of the spine and under the breasts (in older girls). In any one of these areas, candidiasis may appear as itchy areas of moist, crusted skin, sometimes with bright-red patches that may become infected with pus.

Systemic yeast infection usually presents as fever and chills unresponsive to antibacterial therapy and may manifest itself as renal or hepatosplenic infection, meningitis, endophthalmitis, endocarditis, osteomyelitis and/or arthritis
iii. Use of Microbiocidal/Sporicidal Compositions to Prevent or Treat Viral Infection

The microbiocidal compositions of the present invention are useful in the prevention or therapeutic treatment of viral infection, when used in an effective amount. Some viruses kill the cells they infect. Many viral infections may by prevented or treated with the microbiocidal compositions of the present invention, including, but not limited to, e.g., Smallpox (variola): influenza; measles; mumps: polio; chickenpox (varicella); rabies; German measles (rubella): hepatitis A and B; Japanese encephalitis: Herpes Simplex Virus; yellow fever: herpes virus; respiratory viral infections, e.g., common cold, influenza, throat infection (pharyngitis or laryngitis), croup in small children, and inflammation of the windpipe (tracheitis) or other airways (bronchiolitis, bronchitis): hantavirus, Human Immunodeficiency Virus: Epstein-Barr Virus; human T-cell lymphotropic virus type 1 (HTLV-1) arenavirus; and arbovirus.

Many different viruses cause colds. Picornaviruses, such as the rhinoviruses, cause most spring, summer, and fall colds. Influenza viruses and respiratory syncytial viruses, which appear regularly in the late fall and winter, cause a spectrum of illnesses, including colds. Influenza viruses spread easily from person to person in infected droplets that are coughed or sneezed into the air. Rhinoviruses and respiratory syncytial viruses also are spread this way, but perhaps mainly by direct contact with infected secretions carried on the fingers.

The influenza virus very rarely has been associated with inflammation of the brain (encephalitis), heart (myocarditis), or muscle (myositis). Encephalitis may make the person drowsy, confused, or even comatose. Myocarditis may cause heart murmurs or heart failure.

Reye's syndrome is a serious and potentially fatal complication that occurs most commonly in children during epidemics of influenza B, particularly if they have received aspirin or a drug containing aspirin.

The two main types of herpesvirus that cause infections involving blisters on the skin are herpes simplex and herpes zoster. Another herpesvirus, Epstein-Barr virus, causes infectious mononucleosis. Cytomegalovirus, another of the herpesviruses, can produce an illness indistinguishable from infectious mononucleosis. A more recently identified herpesvirus, herpesvirus 6, causes a childhood illness known as roseola infantum. Human herpesvirus 7 has not been definitely linked with any illness at this time. In some studies, herpesvirus 8 has been interpreted to be the cause of Kaposi's sarcoma in people with AIDS.

Herpes simplex infection produces recurring episodes of small, painful, fluid-filled blisters on the skin or mucous membranes. Herpes simplex produces an eruption on the skin or mucous membranes. The eruption subsides, although the virus remains in an inactive (latent) state inside the ganglia (a group of nerve cell bodies) that supply the sensory nerves to the infected area. Periodically, the virus is reactivated and begins replicating, often causing skin eruptions of blisters in the same location as the earlier infection. However, the virus may be present in the skin without causing an obvious blister. The virus in this state can serve as a source for infecting other people. Eruptions may be triggered by overexposure to sunlight, a fever, physical or emotional stress, suppression of the immune system, or certain foods and drugs, but often the inciting factors are unknown. The two types of herpes simplex virus that infect the skin are HSV-1 and HSV-2. HSV-1 is the usual cause of cold sores on the lips (herpes labialis) and sores on the cornea of the eye (herpes simplex keratitis). It is usually transmitted by contact with secretions from or around the mouth. HSV-2 usually causes genital herpes and is transmitted primarily by direct contact with the sores, most often during sexual contact.

The first herpes infection in infants or young children may cause painful sores and inflammation in the mouth and gums (gingivostomatitis) or painful inflammation of the vulva and vagina (vulvovaginitis). These conditions also cause irritability, loss of appetite, and fever. In infants and less often in older children, the infection may spread by way of the blood to involve internal organs, including the brain—an infection that can be fatal.

A woman who has had an HSV-2 infection can transmit the infection to her fetus, especially if an episode occurred in the last 3 months of pregnancy. Herpes simplex virus in a fetus may cause a mild inflammation of the membrane surrounding the brain (meningitis) or occasionally severe brain inflammation (encephalitis).

If infants or adults with a skin condition called atopic eczema become infected with herpes simplex virus, they can develop a potentially fatal illness called eczema herpeticum. Therefore, people with atopic eczema should avoid being near anyone with an active herpes infection. In people with AIDS, herpes infections of the skin may be particularly severe and persistent. Inflammation of the esophagus and intestine, ulcers around the anus, pneumonia, or nerve abnormalities also occur more frequently in people with AIDS.

Shingles (herpes zoster) is an infection that produces a severely painful skin eruption of fluid-filled blisters. Shingles is caused by the same herpesvirus, varicella-zoster virus, that causes chickenpox. The initial infection with varicella-zoster virus, w may be in the form of chickenpox, ends with the virus entering the nerves to the ganglia (a group of nerve cell bodies) of spinal or cranial nerves and remaining latent there. Shingles always is limited to the skin distribution of the nerve root(s) involved (dermatomes).

Pain in areas of skin supplied by the infected nerves is called postherpetic neuralgia. This pain may persist for months or years after an episode of shingles. It does not indicate that the virus continues to be actively replicating. The pain of postherpetic neuralgia may be constant or intermittent, and it may worsen at night or in response to heat or cold. Sometimes the pain is incapacitating. Postherpetic neuralgia occurs most often in older people. 25 to 50 percent of those over age 50 who have shingles also have some postherpetic neuralgia. However, only about 10 percent of all people with shingles develop postherpetic neuralgia, and few have severe pain.

Infectious mononucleosis is a disease characterized by fever, sore throat, and enlarged lymph nodes and is caused by one of the herpesviruses, Epstein-Barr virus. After first invading the cells lining the nose and throat, Epstein-Barr virus spreads to the B lymphocytes (the white blood cells responsible for producing antibodies). Epstein-Barr virus infection is very common, affecting children, adolescents, and adults alike.

The Epstein-Barr virus is associated with Burkitt's lymphoma, a type of cancer that occurs mainly in tropical Africa. The virus also may play a role in certain tumors of B lymphocytes in people with impaired immune systems, such as those with organ transplants or AIDS, and in some cancers of the nose and throat. Although the precise role the Epstein-Barr virus plays in these cancers isn't known, it is thought that specific parts of the virus' genetic material alter the growth cycle of infected cells.

Chronic fatigue syndrome is an illness that occurs mainly among adults aged 20 to 40 years. Twice as many women as men develop chronic fatigue syndrome. Symptoms include debilitating fatigue, interference with the ability to concentrate, and, in some cases, a low-grade fever and swelling of the lymph nodes.

Rabies is a viral infection of the brain that causes irritation and inflammation of the brain and spinal cord. The rabies virus is present in the saliva of infected animals. An animal with rabies transmits the infection to other animals or humans by biting and sometimes by licking. The virus travels from the site of initial inoculation along the nerves to the spinal cord and the brain, where it multiplies. It subsequently travels down nerves to the salivary glands and into the saliva.

The infection is caused by the human T-cell lymphotropic virus type I (HTLV-I). This virus, a retrovirus, also can cause a type of leukemia. Tropical spastic paraparesis may be spread by sexual contact or by contaminated needles. It can also be transmitted from mother to child either across the placenta or in breast milk. The symptoms may begin years after the initial infection. In the process of responding to infection with HTLV-I, the immune system may injure nerve tissue, causing the symptoms. Weakness and muscle stiffness in both legs begin gradually and worsen slowly. Some sensation in the feet may be lost.

Arbovirus is a term used for a virus that is spread to humans by bites from insects, such as ticks and mosquitoes that have become infected by infected animals, including domestic animals and birds. Arbovirus encephalitis is a severe infection of the brain caused by one of several viruses. The most common types of viral encephalitis transmitted by insect bites in the United States are western equine encephalitis, eastern equine encephalitis, St. Louis encephalitis, and California encephalitis. The virus responsible for each of these infections is spread by a specific mosquito type found in particular geographic area. The diseases are endemic zoonoses in the region, but outbreaks occur periodically when the population of infected animals increases.

In other parts of the world, different but related arboviruses that cause encephalitis are transmitted periodically from nature to man. Such diseases include Venezuelan equine encephalitis, Japanese encephalitis, Russian spring-summer encephalitis, and other types of encephalitis named for the geographic area in which they occur. One of the most recognized and historically important arbovirus infections is the one designated as yellow fever. Yellow fever, a viral disease transmitted by mosquitoes, results in fever, bleeding, and jaundice, and it can be fatal. The disease is most common in Central Africa and Central and South America.

Dengue fever is one of the most prevalent arbovirus infections that occurs worldwide in the tropics and subtropics. The infection, transmitted by mosquitoes, results in fever, lymph node swelling, and bleeding. It causes severe joint and muscle pains and is sometimes called breakbone fever. It can be fatal.

Arenaviruses and some viruses related to the arboviruses are viruses that can spread to humans by exposure to rodents or aerosols originating from their droppings. Lymphocytic choriomeningitis is an arenaviral disease that usually produces an influenza-like illness.

The arenavirus that causes lymphocytic choriomeningitis is common in rodents, especially the gray house mouse and the hamster. These animals are usually infected by the virus for life and excrete it in urine, feces, semen, and nasal secretions. Exposure to contaminated dust or food is usually responsible for infection in people.

Ebola and Marburg are two complex viruses of Africa classified as filoviruses. They cause severe hemorrhagic fevers in humans. The Ebola virus probably originates in monkeys. It is often transmitted among humans by exposure to blood or infected body tissues. The infection results in fever, diarrhea, bleeding, and loss of consciousness. It is often fatal, but less virulent strains of the virus may exist. It occurs mainly in East, South, and Central Africa. The Marburg virus is acquired from exposure to infected primate tissues. The virus is highly infectious, causing severe disease that affects many organs. Without treatment, death is almost always inevitable.

Lassa fever is an arenaviral infection transmitted from rodents to humans or from human to human, which results in fever, vomiting, and bleeding. It is highly fatal and requires strict isolation of cases. It occurs mainly in West Africa.

Hantavirus infection is a viral disease that is spread from rodents to humans and causes severe infections of the lungs and kidneys. Hantaviruses are bunyaviruses distantly related to the California group of encephalitis viruses. Hantaviruses are present throughout the world in the urine, feces, and saliva of various rodents, including field and laboratory mice and rats. People acquire the infection by having contact with rodents or their droppings, or possibly by inhaling virus particles in the air.

Human Immunodeficiency Virus is the virus that causes Acquired Immune Deficiency Syndrome (AIDS). HIV is a retrovirus that infects several kinds of cells in the body, the most important of which is a type of white blood cell called the CD4 lymphocyte (or “T cell”). The CD4 cell is a major component of the human immune system that helps keep people free from many infections and some cancers. HIV can effectively disable the body's immune system, and destroy its ability to fight certain diseases. Two major types of HIV have been identified so far. HIV-1 is the cause of the worldwide pandemic, and at least ten different subtypes of HIV-1 have also been found. HIV-2 is found mostly in West Africa HIV is spread through exposure to semen and vaginal fluid (including menstrual blood) from unprotected sex (without a condom) or through exposure to blood from injection drug use via contaminated needles or syringes. HIV can also be transmitted from mother to child during birth or by breastfeeding.
iv. Use of the Microbiocidal/Sporicidal Compositions to Prevent or Treat Dermatitis

The microbiocidal or sporicidal compositions of the present invention are useful in the prevention or therapeutic treatment of dermatitis, when used in an effective amount. Dermatitis is not a single disease, rather the name covers those skin conditions in which inflammation is the key feature. As a result of inflammation, symptoms such as itching are common. Dermatitis is also called eczema, from the Greek ekzein, which means “to boil over or break out,” because of the small blisters (i.e., vesicles) that occur. The chief signs of dermatitis are redness (erythema), a rash (dry flaky skin with small blisters), and pain or itching. Types of dermatitis include:

### Atopic Dermatitis

In atopic dermatitis, skin involvement is symmetrical, i.e., the eczema is distributed equally on each side of the body. In infants, it's seen mainly on the head, face, especially the cheeks, and the outside surfaces of the arms and the front of the legs, particularly the elbows and knees.

The infant's rash is typically dry, with small, raised bumps (papules). In older children, the skin changes are situated more on the limbs than the head, and they tend to show signs of chronic dermatitis (e.g., excoriation, lichenification, fissures) and evidence of infection.

### Contact Dermatitis (Allergic and Irritant Dermatitis)

Unlike atopic dermatitis, contact dermatitis develops at the site at which the culprit substance made direct contact with the skin. Depending on the contact, the distribution may or may not be symmetrical. If the substance is airborne, the distribution will appear on exposed skin areas, such as the face and the backs of the hands. The rash can entirely cover both hands, as is the case in latex glove dermatitis sometimes seen in nurses, maybe with a small strip of normal skin where the person wore a ring. Substances are more easily absorbed where the skin is thinnest, so the backs of the hands are more easily affected than the palms, which have a thicker epidermis. The absorption of chemical through the skin is increased by moisture. Thus, parts of the body where sweat accumulates, such as the axilla, groin and knee flexure, are more likely to be affected.
v. Use of the Microbiocidal/Sporicidal Compositions to Prevent or Treat Inflammatory Disorders and Diseases

The microbiocidal or sporicidal compositions of the present invention can be used to prevent or treat the bacterial, fungal, and/or viral infections that can yield the inflammatory responses that underlie or contribute to inflammatory disorders and disease. As such, the microbiocidal or sporicidal compositions of the present invention are useful in the prevention or therapeutic treatment of inflammatory disorders and diseases, when used in an effective amount.

Inflammation is the body's response to injury, infection or molecules perceived by the immune system as foreign. Clinically, inflammation is characterized by pain, redness, heat, swelling and altered function of affected tissue. Although the ability to mount an inflammatory response is essential for survival, the ability to control inflammation is also necessary for health. Absent, excessive or uncontrolled inflammation results in a vast array of diseases that includes the highly prevalent conditions of: allergy including, e.g., allergic rhinitis/sinusitis, skin allergies (urticaria/hives, angioedema, atopic dermatitis), food allergies, drug allergies, insect allergies, and rare allergic disorders such as mastocytosis; asthma; arthritis, including, e.g., osteoarthritis, rheumatoid arthritis, and spondyloarthropathies; autoimmune conditions, including, e.g., systemic lupus erythematosus, dermatomyositis, polymyositis, inflammatory neuropathies (Guillain Barré, inflammatory polyneuropathies), vasculitis (Wegener's granulomatosus, polyarteritis nodosa), and rare disorders such as polymyalgia rheumatics, temporal arteritis, Sjogren's syndrome, Bechet's disease, Churg-Strauss syndrome, and Takayasu's arteritis; cardiovascular inflammation; gastrointestinal inflammation; infection and immunity; neuroinflammatory disorders and transplantation.

D. Therapeutic Effects and Endpoints

A therapeutically-effective amount is the minimal amount of the active antimicrobial composition that is necessary to impart therapeutic benefit to a subject treated with the antimicrobial composition of the present invention. For example, a therapeutically-effective amount is such an amount which induces, ameliorates or otherwise causes an improvement in the pathological symptoms, disease progression, physiological conditions associated with or resistance to succumbing to a disorder principally characterized by a microbial infection. This includes the microbial infection itself, as well as secondary disorders resulting from or exacerbated by the microbial infection, such as septicemia, inflammation and the like. A prophylactically effective amount is the minimal amount of the active antimicrobial composition, which is necessary to impart a prophylactic benefit to a subject treated with the antimicrobial composition, i.e., prevention of pathological symptoms or resistance to succumbing to a disorder principally characterized by a microbial infection including secondary disorders resulting from or exacerbated by the microbial infection.

### EXPERIMENTAL EXAMPLES

The following examples are intended to be non-limiting illustrations of certain embodiments of the present invention. All references cited are hereby incorporated herein by reference in their entireties.

Industrial application of the proposed preparation is confirmed by Examples 1-8, as follows.

### Example 1

2.0 g of sodium hydroxide is dissolved in 50 cm³ of distilled water in a flask and 3.75 g of glycine is added with stirring. 6.8 g of zinc chloride is added portion-wise to the obtained solution on stirring, followed by the addition of 3.75 cm³ of 25% aqueous solution of ammonium. Separately there is prepared a solution of 0.43 g of cetyltriethylammonium chloride in the mixture of 1.2 g of triethyleneglycol and 15.3 cm³ of water. Both solutions are mixed and diluted with water to achieve the concentration that is required for the antibacterial treatment of objects.

### Example 2

25 ml of water and 11.85 g of ethylenediaminotetraacetic acid are added to 6.1 cm³ of 25% solution of ammonia in a flask. On stirring, there is added portion-wise 5.45 g of copper dichloride, and then 2.4 g of ethanolamine is added. The formed solution turns dark blue. Separately there is prepared a solution of 8.1 g of dodecylbenzyltrimethylammonium chloride in a mixture of 7.3 cm³ of isopropyl alcohol and 10 cm³ of water. Both solutions are mixed and diluted to achieve the concentration required for the antibacterial treatment of objects.

### Example 3

0.4 g of sodium hydroxide is dissolved in 20 cm³ of distilled water in a flask, and 1.46 g of L-lysine is added with stirring. 1.36 g of zinc chloride is then added portion-wise with stirring. The obtained solution is mixed with 0.75 cm³ of a 25% solution of ammonium in water. Separately, a solution of 12.0 g of cetylpyridinium chloride in 56.0 cm³ of isopropyl alcohol is prepared. An aqueous solution of a zinc amino acid complex is added slowly, portion-wise. The mixture is stirred and diluted with water to achieve the concentration that is required for the antibacterial treatment of objects.

### Example 4

A chelating metal complex compound containing a monodentate ligand that displays affinity towards hydrogen ion is mixed with an ionogenic surfactant, as is indicated in Example 1. Distilled water is added to achieve a 10% or 30% concentration, i.e., a ratio with the solvent of 1-9 or 7.

### Example 5

The ingredients are mixed as described in Example 2 in the following amounts (weight %):

| | |
|---|---|
| Chelating metal complex compound containing a monodentate, | 30 |
| bidentate or polydentate ligand that displays affinity towards | |
| hydrogen ion | |
| Ionogenic surfactant | 15 |
| Aliphatic alcohol (C₁-C₈) | 0.5 |
| Distilled water | 54.5 |

### Example 6

The ingredients are mixed as is described in Example 2 in the following amounts (weight %):

| | |
|---|---|
| Chelating metal complex compound containing a monodentate, | 2 |
| bidentate or polydentate ligand that displays affinity towards | |
| hydrogen ion | |
| Ionogenic surfactant | 1 |
| Aliphatic alcohol (C₁-C₈) | 95 |
| Distilled water | 2 |

### Example 7

The ingredients are mixed as is described in Example 3 in the following amounts (weight %):

| | |
|---|---|
| Chelating metal complex compound containing a monodentate, | 1 |
| bidentate or polydentate ligand that displays affinity towards | |
| hydrogen ion | |
| Ionogenic surfactant | 5 |
| Aliphatic alcohol (C₁-C₈) | 20 |
| Distilled water | 74 |

### Example 8

The ingredients are mixed as is described in Example 3 in the following amounts (weight %):

| | |
|---|---|
| Chelating metal complex compound containing a monodentate, | 2 |
| bidentate or polydentate ligand that displays affinity towards | |
| hydrogen ion | |
| Ionogenic surfactant | 0.1 |
| Aliphatic alcohol (C₁-C₈) | 30 |
| Distilled water | 67.9 |

### Example 9

### Measuring the Disinfecting Property of the Antimicrobial Composition (AMC) Against Select Bacteria, Yeast and Fungi Using the Time-Kill Test

### I. General

The antimicrobial product (hereinafter referred to as “AMC”) is a microbiocidal and sporicidal composition of the present invention that contains the following synergistic active ingredients: Isopropanol (“IPA”, CAS No. 67-63-0); Zinc (“Zn”) complexed with ethylene diamine tetra-acetic acid (EDTA, CAS No. 60-00-4); and Cetyl Pyridinium Chloride (“CPC”, CAS No. 123-03-5).

As demonstrated below, this microbiocidal and sporicidal composition provides a degree of killing of fungal organisms not seen with either alcohol preparations or quaternary ammonium compounds alone and displays higher potency for fungicidal activity than typically observed with zinc-based products.

AMC is a liquid concentrate product of nearly neutral pH (ca. 7.5) that contains 49% IPA and 49% water. Its make-up and chemical and physical characteristics indicate that it can be formulated into various product forms such as, e.g., but not limited to, aqueous dilution to provide a working antimicrobial solution for various applications; formulation into hydrogel ointments; formulation into water soluble creams; addition to other liquid, gel or cream products to provide a preservative function for these products; pre-packaged as a ready-to-use dilution for specific applications; pre-packaging into disinfectant wipes; or, pre-packaging into disinfectant bandages and dressing.

A panel of “challenge microorganisms,” including select bacteria, yeast, and fungi was used to assess the disinfecting properties of test compounds, e.g., AMC (DPT Laboratories). Specifically, the disinfectant property of test compounds were tested against Staphylococcus aureus (ATCC 25923); Staphylococcus epidermidis (ATCC 12228); Bacillus subtilis (ATCC 19659); Escherichia coli (ATCC 11229); and Pseudomonas aeruginosa (ATCC 15442) bacteria, as well as the yeast Candida albicans (ATCC 10231), and the fungus Trichophyton rubrum (ATCC 28188). The test procedure incorporated the recommendations described in the “Manual of Clinical Microbiology,” 5th ed., edited by A. B. Balows et al., ASM, Washington, as directed by the Federal Register, June 1994. The procedure was based on the ASTM procedure entitled, “Standard Test Method for the Assessment of Microbial Activity of Test Materials Using Time-Kill Procedure.”

It is important that disinfecting compositions, e.g., skin antiseptic preparation, provide rapid and prolonged antimicrobial action. The Time-Kill test evaluates the rapidity of the antimicrobial action, whereas the Minimum Inhibition Concentration assesses the prolonged inhibiting action.

### II. Test Conditions

A single lot of test material was prepared at various concentrations, along with multiple compounds prepared in various concentrations and combinations. These test materials were tested in duplicate against various bacteria, yeast and fungi. To minimize potential buffer interference and to minimize reduction of antimicrobial activity, the volume of the inoculum added to the test material was maintained at, or below, 1% of the total volume of the test. Samples were removed at various contact times. Serial dilutions were performed, and duplicate aliquots were plated. The plates were then incubated, and the average colony forming units (CFU) recovered per milliliter were determined for each contact time. The contact times for the test compounds with S. aureus, E. coli, P. aeruginosa, and C. albicans were 30 sec, 1 min, and 5 min. The contact times for the test compounds with B. subtilis were 1 and 10 min, 1 h, 2 h, 3 h, and 24 h. The contact times for the test compounds with T. rubrum were 30 sec, 1 min and 10 min. The contact temperature was ambient room temperature (20-21° C.).

### III. Experimental Design

A. Inocula Preparation
1. Bacteria and Yeast

Bacteria and C. albicans from stock cultures were transferred into trypticase soya broth (TSB) and incubated for 18-24 hours at 37° C.±2° C. A second transfer was made onto Trypticase Soya Agar (TSA). The plate was removed from incubation and the growth was washed from the agar surface with Butterfield's Phosphate Buffered Dilution Water (PBDW). The microbe concentration was measured using a spectrophotometric technique well-known in the art. The suspension was subsequently adjusted to contain approximately 10⁸ colony forming units (CFU) per ml.
2. Fungus

The fungus, T. rubrum, was inoculated onto Emmon's agar (EA) and incubated at 25-30° C. for 10-15 days. The mycelial mats from mature cultures were removed from the surface of at least 5 plates and macerated with sterile saline (SS) in a sterile glass tissue grinder. The suspension was filtered through sterile glass wool to remove the hyphae.

The density of the conidial suspension was determined by serially diluting the prepared culture in BPDW. Aliquots from selected dilutions were plated on duplicate EA plates. The plates were incubated for 3-5 days at 25-30° C. and then each plate was examined for enumeration.

The suspension was stored at 2-10° C. for 4 weeks before use. On the day of testing, the suspension was adjusted to yield approximately 5.0×10⁸ CFU by dilution with BPDW.

B. Preparation of the Test Material

AMC was assessed for disinfecting activity at the indicated concentrations. The active ingredients of AMC, e.g., cetylpyridinium chloride (CPC) and ZNEDTA, were tested alone, and in combination with other agents to determine synergy of the components with regard to the disinfecting activity. The combinations of components tested were as follows:
Synergy of 0.2% CPC and Other AMC Components:
   ZnEDTA (1%), CPC (0.2%), and isopropyl alcohol (9.8%)
   ZnEDTA (1%), and CPC (0.2%)
   Isopropyl alcohol (9.8%) and CPC (0.2%)
Synergy of 0.02% CPC and Other AMC Components:
   ZnEDTA (1%), CPC (0.02%), and isopropyl alcohol (9.8%)
   ZnEDTA (1%), and CPC (0.02%)
   Isopropyl alcohol (9.8%) and CPC (0.02%)
Synergy of 0.002% CPC and Other AMC Components:
   ZnEDTA (1%), CPC (0.002%), and isopropyl alcohol (9.8%)
   ZnEDTA (1%), and CPC (0.0020%)
   Isopropyl alcohol (9.8%) and CPC (0.002%)

Other control compounds were tested individually including Hibiclens (100%; active ingredient: chlorhexidine gluconate); Ciprofloxecin; Betadine (100%; active ingredient: providine iodine); 1% ZnEDTA; cetylpyridinium chloride (CPC) at 0.2%, 0.02%, and 0.002% concentrations; 9.8% isopropyl alcohol; Miconazole (100%); Lamisil; Fluconazole (2 mg/ml; C. albicans only); and Amphotericin B (2.5 mg/L; T. rubrum only).

C. Time Kill Test

Compliant with the ASTM procedure cited above, the challenge microorganism was added to the test material by dispensing 99 ml of the test material into two sterile flasks, each containing a stir bar. The reaction flasks were allowed to equilibrate to the test temperature for at least 10 minutes. The flasks were placed in water baths on stir plates and maintained at the test temperature with stirring. A 1 ml aliquot of the prepared inoculum of the challenge microorganism was added to each flask to begin the contact period. At each contact time, 1 ml aliquot samples were removed and added to tubes containing 9 ml PBDW+. Serial 10-fold dilutions of each sample were then prepared in PBDW dilution blanks. Duplicate aliquots from selected dilutions were plated using the appropriate agar.

D. Incubation and enumeration

Upon completion of all contact periods, all plates were inverted and incubated for the appropriate time and temperature as follows: bacteria and yeast were incubated over 2 nights at 37° C.±2° C., and the fungus was incubated for at least 5 nights at 25-30° C. Following incubation, all plates were removed, the colonies were counted and the CFU recovered per ml at each contact time were calculated.

E. Controls
1. Initial Counts

For each challenge microorganism, 99 ml of PBDW was dispensed into a sterile flask, and a 1 ml aliquot of prepared inoculum was added to the flask with stirring. Within 30 sec, a 1 ml sample was removed and serial 10-fold dilutions were performed in PBDW dilution blanks. Duplicate aliquots from selected dilutions were plated using the appropriate agar. Plates were inverted and treated in the same manner as the test plates (see Incubation and enumeration above). The procedure was performed for each challenge microorganism at the initiation of testing.
2. Neutralizer effectiveness

This control was included to establish the usefulness of the neutralizer. This procedure was performed using a Gram-positive bacterium; a Gram-negative bacterium; and a fungus. For each microorganism, 4 tubes were prepared with 9 ml PBDW+ and fewer than 100 CFU of the microorganism. To each tube, 1 ml of test material was added. Immediately, the entire contents of 2 tubes were filtered using a microbiological filtration technique well-known in the art, and the filters were retained. The remaining tubes were held at room temperature for 30 min., and the entire contents of the remaining tubes were filtered. All of the filters were placed on agar appropriate to the microorganism under analysis. The CFU added to each tube was plated in duplicate using the appropriate agar. The plates were treated in the same manner as the test plates appropriate to the microorganism under analysis. The neutralizers used in these studies were PBDW containing 1% glycine, 7% Polysorbate 80, and 1% lecithin (used for evaluation of Gram-positive and Gram-negative bacteria testing with AMC; also using fungi, the prepared 20% AMC and 2.5 mg/L Amphotericin B test agents); PBDW containing 7% Polysorbate 80 and 1% lecithin (used for evaluation of Hibiclens); PBDW containing 0.3% Na₂S₂O₃ (used for evaluation of Betadine); and PBDW.
3. Sterility Control

Duplicate plates of each agar type were incubated with the test material. In addition, 1 ml aliquots of PBDW and PBDW+ were plated in duplicate using one of the agar types used for the test. These plates were incubated with the bacteria and the yeast plates.
4. Organism Confirmation

In order to confirm growth consistent with the challenge microorganisms, Gram stains were performed from a representative colony on an initial count control plate for all bacteria and yeast. The colony morphology was noted. The fungus was confirmed through wet mount observation and the morphology was documented. Where appropriate, an isolated colony from a test plate was treated in the same manner and compared to the initial count control stain or wet mount.

### IV. Experimental Results

A. Initial Studies

Time-kill tests were used to measure the disinfecting property of test compounds, e.g., microbiocidal or sporicidal compositions, using select bacterial challenge microorganisms such as the vegetative forms of, e.g., E. coli (strain 1257) S. aureus (strain 906), and Bacillus cereus (strain 96). The results of the initial studies are summarized below in Table 1.

TABLE 1
Antimicrobial activity of samples

| | | | Death time of test- | | |
|---|---|---|---|---|---|
| | | | microorganisms (min) | | |
| No | | | E. | | |
| n/n | Sample | Conc. (%) | coli | S. aureus | B. cereus. |
| 1 | Ethylenediamino- | 0.1 | >30 | >30 | — |
| | tetraacetate zinc complex | 5.0 | >30 | >30 | |
| 2 | Monoglycinatecopper | 0.1 | >30 | >30 | — |
| | chloride complex | 0.2 | 30 | >30 | — |
| | | 0.5 | 15 | >30 | — |
| | | 5.0 | | >30 | >360 |
| 3 | Preparation 1 on the | 0.025 | 30 | >30 | — |
| | basis of glycinatecopper | 0.05 | 5 | 5 | — |
| | chloride complex | 2.0 | 5 | 5 | <60 |
| 4 | Preparation 2 on the | 0.05 | 5 | 5 | — |
| | basis of ethylenediamino- | 0.1 | 5 | 5 | — |
| | tetraacetate zinc complex | 5.0 | 5 | 5 | <60 |

As shown in Table 1, preparations of chelating metal complexes, e.g., glycinatecopper ammonium chloride, were bactericidal towards the vegetative challenge organisms tested. In order to increase the disinfecting property, e.g., microbiocidal and sporicidal activity of the chelating metal complex test preparations, ionogenic surfactants (cetylpyridinium chloride, cetyltrimethylammonium bromide) were added. This composition (preparation 1, Table 1) displayed bactericidal activity toward both Gram-negative and Gram-positive challenge microorganisms consistent with synergy between the glycinatecopper ammonium chloride and cetyltrimethylammonium bromide components of the preparation. Preparation 2 contained 2-aminoethanol diaminotetraacetate zinc complex and cetylpyridinium chloride. This test preparation showed the highest level of bactericidal activity toward the challenge organisms tested.

A test preparation containing 5% solution of ethylenediaminotetraacetate zinc complex in a water-alcohol solution (70 vol. % isopropyl alcohol) showed bactericidal activity towards vegetative types of bacteria even at a 128-fold dilution of the stock preparation. Further, this test preparation displayed sporicidal activity towards B. cereus at a 16-fold dilution of the stock preparation. Bacillus anthracis, a Gram-positive spore-forming soil bacillus, is a member of the B. cereus group species, B. cereus and B. thuringiensis. These species are very similar physiologically and genetically, yet they cause vastly different diseases. Therefore, B. cereus has been used as a less harmful surrogate model organism for studies relevant to the biology of B. anthracis.

As noted above, the proposed universal, ecologically safe bactericidal preparation is intended for disinfecting the main forms and types of pathogenic microflora, including the spore form. The preparation exhibits increased ecological properties that are achieved by applying nontoxic chelating agents and transforming metal ions into nontoxic chelating complexes. Advantages of this preparation include: 1. a reduced cost of the bactericide complex; 2. an increased environmental stability of the bactericide due to the increased independence of the preparation from various environmental factors, e.g., temperature, humidity, and light effect; and 3. retention of disinfecting property for long periods, e.g., one year or more.

The results of these initial studies showed the disinfecting property of the test preparations toward a variety of bacterial strains. Based on these findings, these preparations are effective against pathogens that cause intestinal infections (Gram-negative bacteria), e.g., P. aeruginosa, dysentery and salmonellosis; respiratory tract and hospital infections (Gram-positive bacteria), e.g., Staphylococcosis, Streptococcosis, and microflora; Anaerobic infections e.g., wound infections (tetanus); and Anthrax (spores). The preparation is also a viricide and disinfects viruses (e.g., hepatitis, herpes, AIDS-infection, rotaviral infections).

B. Time Kill Study 509-101

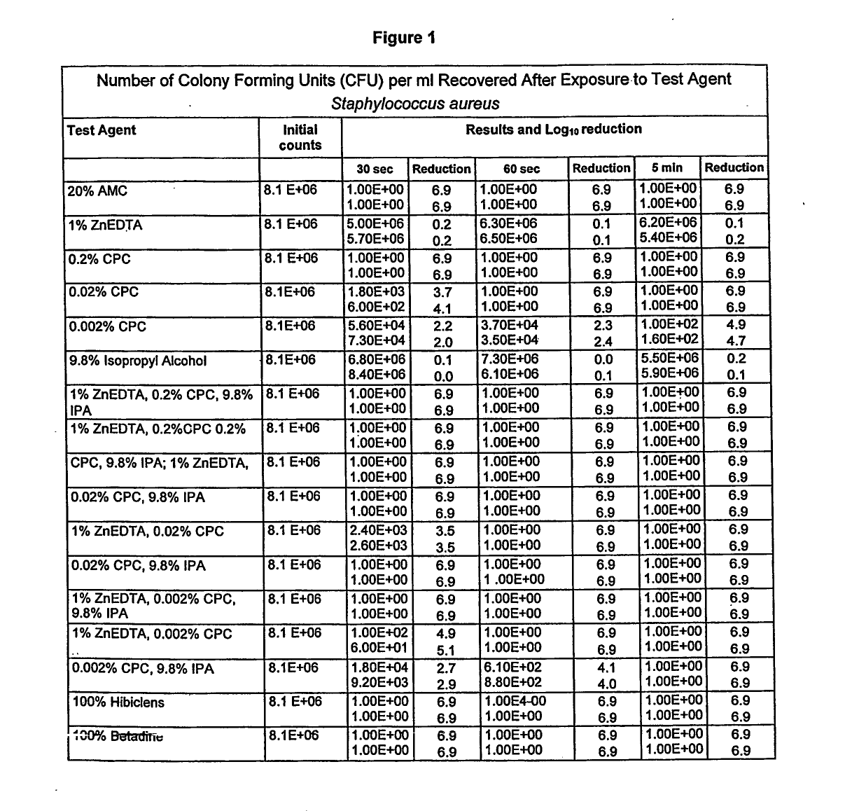

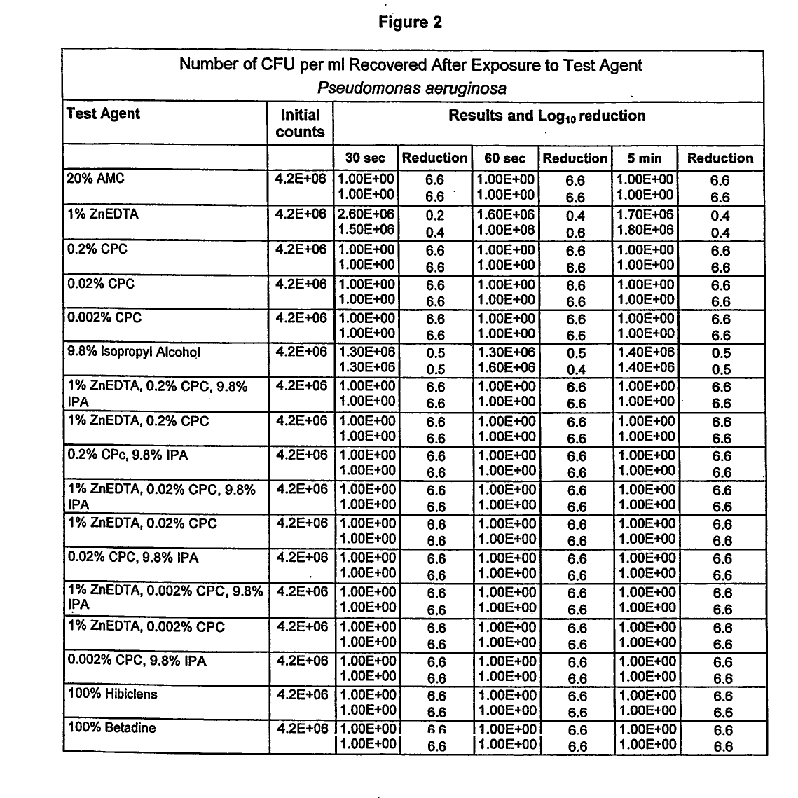

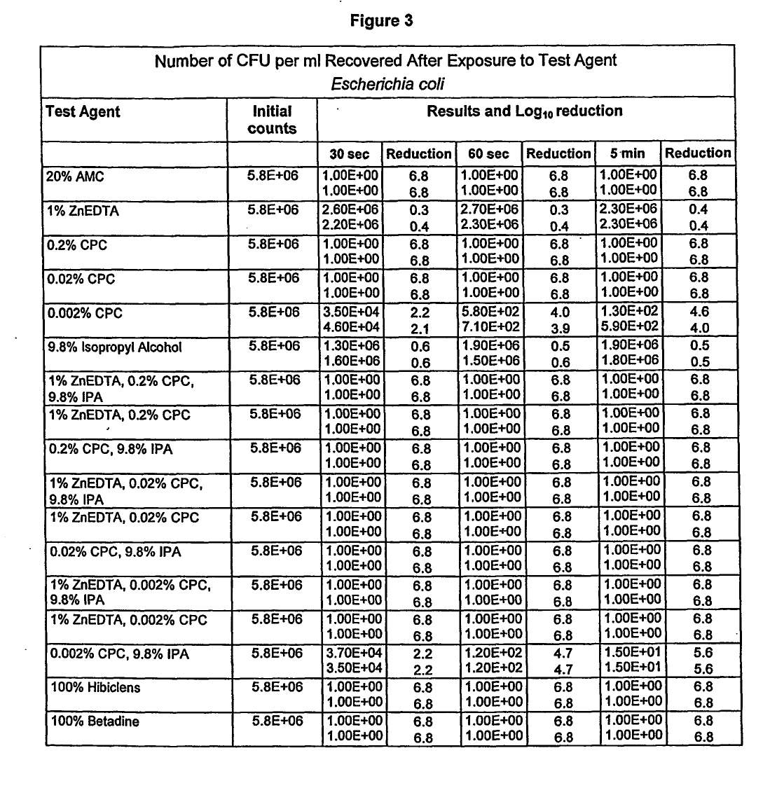

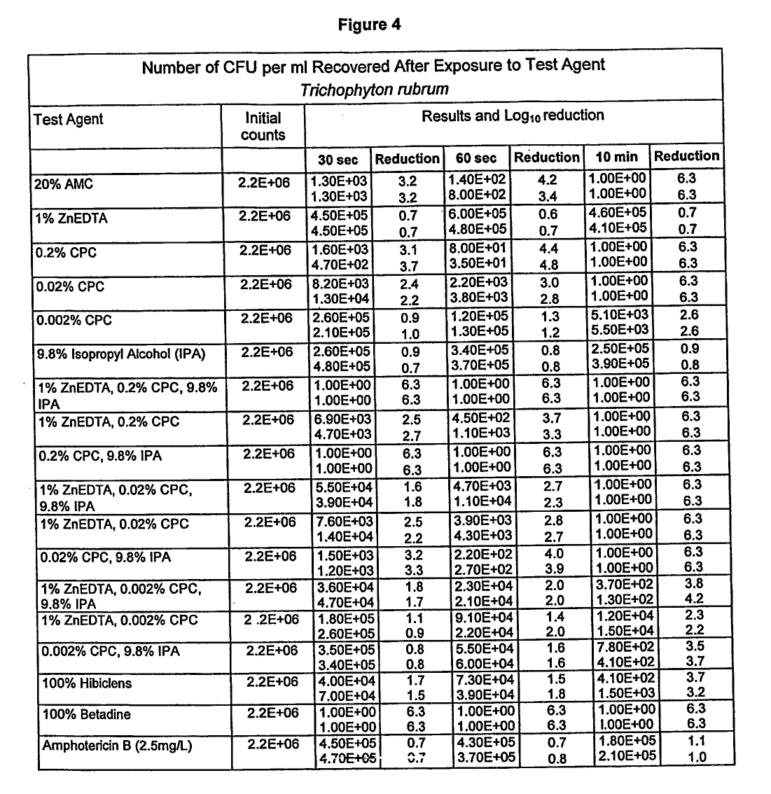

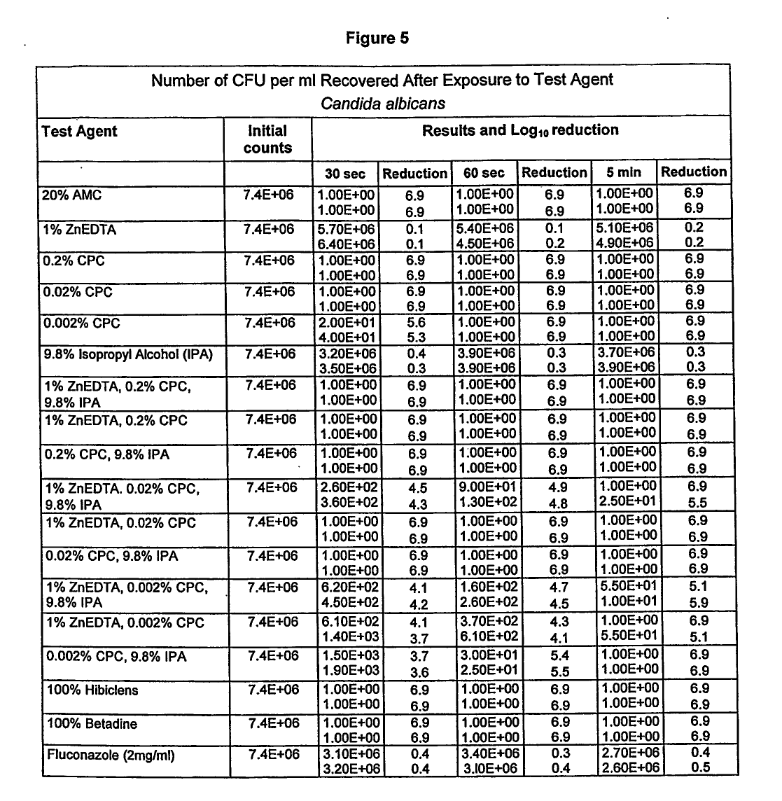

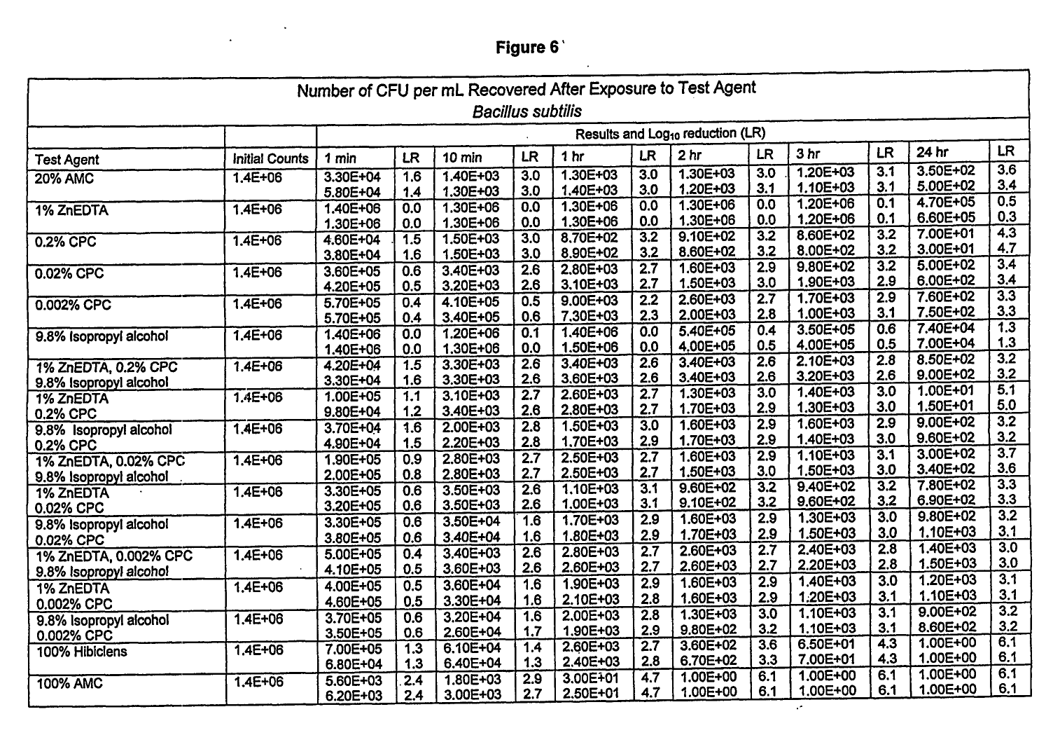

Results for Time Kill Study 509-101 are presented in FIGS. 1-7 below. Each test agent was evaluated in duplicate using various concentrations and combinations of test agents. Log₁₀ reduction was calculated using the following equation:

Log₁₀(Initial counts control)−Log₁₀(Test result)=Log₁₀ Reduction

### Counts of <5.0 colony forming units (CFU) per ml are reported as 1.0 for calculation purposes.

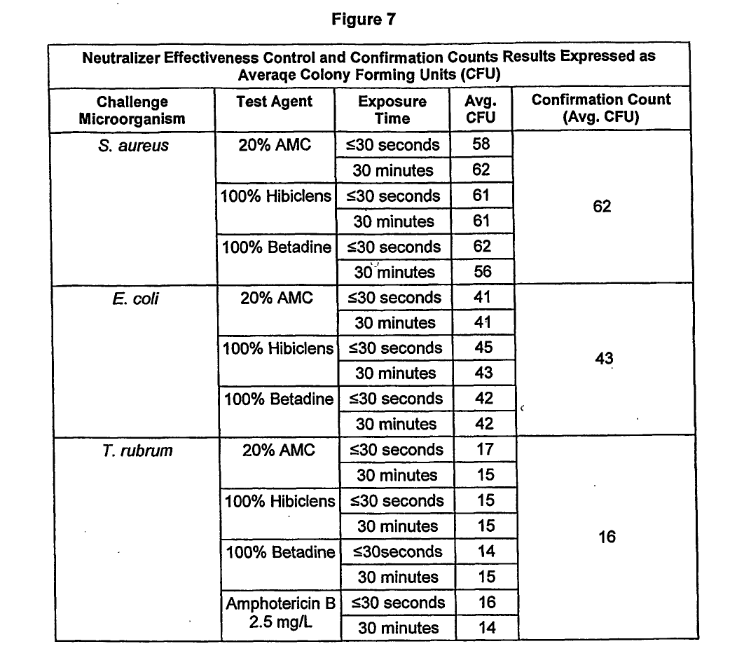

The effectiveness of each neutralizer was validated with comparable recovery between the zero (≦30 seconds) and thirty minute exposure times, as indicated in FIG. 7. The sterility controls exhibited no growth. The challenge microorganisms were confirmed by Gram stain or wet mount and colony morphology.

As shown in FIGS. 1-7, when tested as described, 20% AMC, and CPC at various concentrations (0.2, 0.02, and 0.002%) demonstrated quick kill when challenged by E. coli, P. aeruginosa, and C. albicans. Although 0.002% CPC reduced S. aureus by 2 log₁₀ at 30 seconds and 1% ZnEDTA was ineffective, when used in combination, they reduced the count approximately 5 log₁₀. All concentrations of CPC were effective against P. aeruginosa, and 0.2% and 0.02% concentrations were effective against E. coli and C. albicans. When individually tested, 1% ZnEDTA, 9.8% isopropyl alcohol and 0.2% CPC were not effective against T. rubrum in 30 sec; however, when used in combination, they reduced the count 6 log₁₀ in 30 sec. A 6 log₁₀ reduction was not achieved when 20% AMC, CPC at various concentrations (e. g., 0.2, 0.02, and 0.002%), and 1% ZnEDTA, and 9.8% alcohol were challenged individually and in combinations with B. subtilis. The test agent and various concentrations of the active ingredient, CPC, were effective antimicrobials and exhibit synergistic effects in combination with 1% ZnEDTA and 9.8% isopropyl alcohol. All of the control cultures met the criteria established for a valid test.

B. Time Kill Study 509-102

Table 2 below indicates the stock dilutions for each test agent, and a breakdown of the resulting concentrations for each of the doubling dilutions performed is summarized in Table 3.

TABLE 2

| | | Starting | | | |
|---|---|---|---|---|---|
| DS | Test Material | Concentration | | Stock Dilution | |
| No. | (Name Only) | of Active | Test Dilution | Concentration | |
| 6303 | AMC | 1% | 20%-0.002% | 20 | ppm |
| 6253 | Hibiclens | 4% | 100%-4% | 40,000 | ppm |
| 6258 | Amphotericin B | 250 μg/mL | 2.5 mg/L | 2.5 | ppm |
| 6356 | Ciprofloxacin | 809 μg/mg | 1 μg/mL | 1 | ppm |
| 6353 | Miconazole | 100% | 20 mg/mL | 20 | ppm |

From the stock dilutions, an initial 1:10 dilution was made (representing tube 1), followed by doubling dilutions (tubes 2-12), as detailed in Table 3.

TABLE 3

| | Resulting Concentration | | | | |
|---|---|---|---|---|---|
| Tube No. | AMC | Hibiclens | Amph. B | Cipro | Micon. |
| 1 | 2 ppm | 4,000 ppm | 0.25 ppm | 0.1 ppm | 2 ppm |
| 2 | 1 ppm | 2,000 ppm | 0.125 ppm | 0.05 ppm | 1 ppm |
| 3 | 0.5 ppm | 1,000 ppm | 0.063 ppm | 0.025 ppm | 0.5 ppm |
| 4 | 0.25 ppm | 500 ppm | 0.031 ppm | 0.0125 ppm | 0.25 ppm |
| 5 | 0.125 ppm | 250 ppm | 0.016 ppm | 0.006 ppm | 0.125 ppm |
| 6 | 0.063 ppm | 125 ppm | 0.008 ppm | 0.003 ppm | 0.063 ppm |
| 7 | 0.031 ppm | 62.5 ppm | 0.004 ppm | 0.0016 ppm | 0.031 ppm |
| 8 | 0.016 ppm | 31.25 ppm | 0.002 ppm | 0.0008 ppm | 0.016 ppm |
| 9 | 0.008 ppm | 15.63 ppm | 0.001 ppm | 0.0004 ppm | 0.008 ppm |
| 10 | 0.004 ppm | 7.81 ppm | 0.0005 ppm | 0.0002 ppm | 0.004 ppm |
| 11 | 0.002 ppm | 3.91 ppm | 0.00025 ppm | 0.0001 ppm | 0.002 ppm |
| 12 | 0.001 ppm | 1.95 ppm | 0.00013 ppm | 0.00005 ppm | 0.001 ppm |

The MIC was considered to be the concentration of the test compound that inhibited growth of the challenge microorganism (i.e., the least concentrated tube which exhibits no visible growth). The MBC was considered to be the concentration of the test compound that inhibited growth of the challenge microorganism as well, so long as at least a 5.0×10⁵ was achieved on the inoculum counts, i.e., exhibiting at least a 99.9% reduction.

The MIC and MBC determinations for select bacterial strains challenged with AMC, Hibiclens and Ciprofloxacin are summarized in Tables 4-8. Specifically, the MIC and MBC values observed for P. aeruginosa are summarized in Table 4 below.

TABLE 4
Pseudomonas aeruginosa - Inoculum Counts: 8.8 × 10⁵

| Test Agent Name | MIC (ppm) | MBC (ppm) |
|---|---|---|
| AMC | 2.0 | 2.0 |
| Hibiclens | 125 | 125 |
| Ciprofloxacin | >0.1 | >0.1 |

### The MIC and MBC values observed for S. aureus are summarized in Table 5 below.

TABLE 5
Staphylococcus aureus - Inoculum Counts: 7.5 × 10⁶

| Test Agent Name | MIC (ppm) | MBC (ppm) |
|---|---|---|
| AMC | 0.008 | 0.008 |
| Hibiclens | <1.95 | <1.95 |
| Ciprofloxacin | >0.1 | >0.1 |

### The MIC and MSC values observed for E. coli are summarized in Table 6 below.

TABLE 6
Escherichia coli - Inoculum Counts: 9.4 × 10⁵

| Test Agent Name | MIC (ppm) | MBC (ppm) |
|---|---|---|
| AMC | 0.26 | 0.25 |
| Hibiclens | 3.91 | 3.91 |
| Ciprofloxacin | >0.1 | >0.1 |

### The MIC and MBC values observed for S. epidermidis are summarized in Table 7 below.

TABLE 7
S. epidermidis - Inoculum Counts: 9.0 × 10⁵

| Test Agent Name | MIC (ppm) | MBC (ppm) |
|---|---|---|
| AMC | 0.016 | 0.016 |
| Hibiclens | 3.91 | 3.91 |
| Ciprofloxacin | >0.1 | >0.1 |

### The MIC and MBC values observed for S. pyogenes are summarized in Table 8 below.

TABLE 8
S. pyogones - Inoculum Counts: 9.8 × 10⁵

| Test Agent Name | MIC (ppm) | MBC (ppm) |
|---|---|---|
| AMC | 0.016 | 0.016 |
| Hibiclens | 3.91 | 3.91 |
| Ciprofloxacin | >0.1 | >0.1 |

The MIC and MBC determinations for select yeast and fungal strains challenged with AMC, Hibiclens, Ciprofloxacin, and Miconazole are summarized in Table 9 and Table 10, respectively. Specifically, the MIC and MBC values observed for the yeast, C. albicans, are summarized in Table 9 below.

TABLE 9
C. albicans - Inoculum Counts: 6.6 × 10⁵

| Test Agent Name | MIC (ppm) | MBC (ppm) |
|---|---|---|
| AMC | 0.031 | 0.031 |
| Hibiclens | 7.81 | 7.81 |
| Ciprofloxacin | >0.1 | >0.1 |
| Miconazole | 2.0 | 2.0 |

### The MIC and MBC values observed for T. rubrum are summarized in Table 10 below.

TABLE 10
T. rubrum - Inoculum Counts: 3.6 × 10⁸

| Test Agent Name | MIC (ppm) | MBC (ppm) |
|---|---|---|
| AMC | 0.031 | 0.031 |
| Hibiclens | 31.25 | 31.25 |
| Amphotericin B | >0.25 | >0.25 |
| Ciprofloxacin | >0.1 | >0.1 |
| Miconazole | 0.25 | 0.25 |

C. Time Kill Study 506-103

Results for Time Kill Study 506-103 are summarized in Tables 11-16. Each test compound was evaluated in duplicate using various concentrations and combinations. Log₁₀ reduction was calculated using the following equation:

Log₁₀(Initial counts control)−Log₁₀(Test result)=Log₁₀ Reduction

The initial counts (CFU/ml) for each challenge microorganism are shown in Table 11 below.

TABLE 11

| Challenge microorganism | CFU/ml |
|---|---|
| S. aureus | 1.7 × 10⁷ |
| P. aeruginosa | 5.5 × 10⁶ |
| E. coli | 5.8 × 10⁵ |
| C. albicans | 3.0 × 10⁷ |
| T. rubrum | 1.0 × 10⁶ |
| B. subtilis | 6.2 × 10⁵ |

The number of CFUs recovered per milliliter of S. aureus culture after exposure to select test compounds and Log₁₀ reduction are summarized in Table 12 below.

TABLE 12

| Test Agent | Rep. | 30 sec. | Reduction | 60 sec. | Reduction | 6 min. | Reduction |
|---|---|---|---|---|---|---|---|
| 20% AMC | 1 | 1.0 × 10⁰ | 7.2 | 1.0 × 10⁰ | 7.2 | 1.0 × 10⁰ | 7.2 |
| | 2 | 1.0 × 10⁰ | 7.2 | 1.0 × 10⁰ | 7.2 | 1.0 × 10⁰ | 7.2 |
| 100% Hibiclens | 1 | 1.0 × 10⁰ | 7.2 | 1.0 × 10⁰ | 7.2 | 1.0 × 10⁰ | 7.2 |
| | 2 | 1.0 × 10⁰ | 7.2 | 1.0 × 10⁰ | 7.2 | 1.0 × 10⁰ | 7.2 |
| 100% Betadine | 1 | 6.5 × 10¹ | 5.4 | 1.0 × 10⁰ | 7.2 | 1.0 × 10⁰ | 7.2 |
| | 2 | 3.5 × 10¹ | 5.7 | 1.0 × 10⁰ | 7.2 | 1.0 × 10⁰ | 7.2 |

The number of CFUs recovered per milliliter of P. aeruginosa culture after exposure to select test compounds and Log₁₀ reduction are summarized in Table 13 below.

TABLE 13

| Test Agent | Rep. | 30 sec. | Reduction | 60 sec. | Reduction | 5 min. | Reduction |
|---|---|---|---|---|---|---|---|
| 20% AMC | 1 | 1.0 × 10⁰ | 6.7 | 1.0 × 10⁰ | 6.7 | 1.0 × 10⁰ | 6.7 |
| | 2 | 1.0 × 10⁰ | 6.7 | 1.0 × 10⁰ | 6.7 | 1.0 × 10⁰ | 6.7 |
| 100% Hibiclens | 1 | 1.0 × 10⁰ | 6.7 | 1.0 × 10⁰ | 6.7 | 1.0 × 10⁰ | 6.7 |
| | 2 | 1.0 × 10⁰ | 6.7 | 1.0 × 10⁰ | 6.7 | 1.0 × 10⁰ | 6.7 |
| 100% Betadine | 1 | 1.0 × 10⁰ | 6.7 | 1.0 × 10⁰ | 6.7 | 1.0 × 10⁰ | 6.7 |
| | 2 | 1.0 × 10⁰ | 6.7 | 1.0 × 10⁰ | 6.7 | 1.0 × 10⁰ | 6.7 |

The number of CFUs recovered per milliliter of E. coli culture after exposure to select test agents and Log₁₀ reduction are summarized in Table 14 below.

TABLE 14

| Test Agent | Rep. | 30 sec. | Reduction | 60 sec. | Reduction | 6 min. | Reduction |
|---|---|---|---|---|---|---|---|
| 20% AMC | 1 | 1.0 × 10⁰ | 6.8 | 1.0 × 10⁰ | 6.8 | 1.0 × 10⁰ | 6.8 |
| | 2 | 1.0 × 10⁰ | 6.8 | 1.0 × 10⁰ | 6.8 | 1.0 × 10⁰ | 6.8 |
| 100% Hibiclens | 1 | 1.0 × 10⁰ | 6.8 | 1.0 × 10⁰ | 6.8 | 1.0 × 10⁰ | 6.8 |
| | 2 | 1.0 × 10⁰ | 6.8 | 1.0 × 10⁰ | 6.8 | 1.0 × 10⁰ | 6.8 |
| 100% Betadine | 1 | 1.0 × 10⁰ | 6.8 | 1.0 × 10⁰ | 6.8 | 1.0 × 10⁰ | 6.8 |
| | 2 | 1.0 × 10⁰ | 6.8 | 1.0 × 10⁰ | 6.8 | 1.0 × 10⁰ | 6.8 |

The number of CFUs recovered per milliliter of C. albicans culture after exposure to select test compounds and Log₁₀ reduction are summarized in Table 15 below.

TABLE 15

| Test Agent | Rep. | 30 sec. | Reduction | 60 sec. | Reduction | 5 min. | Reduction |
|---|---|---|---|---|---|---|---|
| 20% AMC | 1 | 1.0 × 10⁰ | 7.5 | 1.0 × 10⁰ | 7.5 | 1.0 × 10⁰ | 7.5 |
| | 2 | 1.0 × 10⁰ | 7.5 | 1.0 × 10⁰ | 7.5 | 1.0 × 10⁰ | 7.5 |
| 100% Hibiclens | 1 | 1.0 × 10⁰ | 7.5 | 1.0 × 10⁰ | 7.5 | 1.0 × 10⁰ | 7.5 |
| | 2 | 1.0 × 10⁰ | 7.5 | 1.0 × 10⁰ | 7.5 | 1.0 × 10⁰ | 7.5 |
| 100% Betadine | 1 | 2.5 × 10¹ | 6.1 | 1.0 × 10⁰ | 7.5 | 1.0 × 10⁰ | 7.5 |
| | 2 | 3.0 × 10¹ | 6.0 | 1.0 × 10⁰ | 7.5 | 1.0 × 10⁰ | 7.5 |
| Fluconazole | 1 | 3.0 × 10⁶ | <1.0 | 3.0 × 10⁶ | <1.0 | 3.0 × 10⁶ | <1.0 |
| | 2 | 3.0 × 10⁶ | <1.0 | 3.0 × 10⁶ | <1.0 | 3.0 × 10⁶ | <1.0 |
| Lamisil | 1 | 3.0 × 10⁶ | <1.0 | 3.0 × 10⁶ | <1.0 | 9.7 × 10⁵ | 1.5 |
| | 2 | 3.0 × 10⁶ | <1.0 | 3.0 × 10⁶ | <1.0 | 1.2 × 10⁶ | 1.4 |

The number of CFUs recovered per milliliter of T. rubrum culture after exposure to select test compounds and Log₁₀ reduction are summarized in Table 16 below.

TABLE 16

| Test Agent | Rep. | 30 sec. | Reduction | 60 sec. | Reduction | 10 min. | Reduction |
|---|---|---|---|---|---|---|---|
| 20% AMC | 1 | 1.0 × 10⁰ | 6.0 | 1.0 × 10⁰ | 6.0 | 1.0 × 10⁰ | 6.0 |
| | 2 | 1.0 × 10⁰ | 6.0 | 1.0 × 10⁰ | 6.0 | 1.0 × 10⁰ | 6.0 |
| 100% Hibiclens | 1 | 3.2 × 10³ | 2.5 | 1.5 × 10³ | 2.8 | 1.0 × 10⁰ | 6.0 |
| | 2 | 1.4 × 10³ | 2.9 | 1.5 × 10³ | 2.8 | 1.0 × 10⁰ | 6.0 |
| 100% Betadine | 1 | 1.0 × 10⁰ | 6.0 | 1.0 × 10⁰ | 6.0 | 1.0 × 10⁰ | 6.0 |
| | 2 | 1.0 × 10⁰ | 6.0 | 1.0 × 10⁰ | 6.0 | 1.0 × 10⁰ | 6.0 |
| Amphotericin B | 1 | 1.0 × 10⁵ | 1.0 | 6.9 × 10⁴ | 1.2 | 9.6 × 10⁴ | 1.0 |
| | 2 | 1.1 × 10⁵ | 1.0 | 8.7 × 10⁴ | 1.1 | 1.0 × 10⁵ | 1.0 |
| Lamisil | 1 | 1.0 × 10⁰ | 6.0 | 1.0 × 10⁰ | 6.0 | 1.0 × 10⁰ | 6.0 |
| | 2 | 1.0 × 10⁰ | 6.0 | 1.0 × 10⁰ | 6.0 | 1.0 × 10⁰ | 6.0 |

The number of CFUs recovered per milliliter of Bacillus subtilis culture after exposure to select test compounds and Log₁₀ reduction are summarized in Table 16a and Table 16b below.

TABLE 16a

| Test Agent | Rep | 30 sec | LR | 10 min | LR | 1 hr | LR |
|---|---|---|---|---|---|---|---|
| 20% AMC | 1 | 3.8 × 10⁵ | 0.2 | 1.8 × 10⁴ | 1.5 | 3.5 × 10³ | 2.2 |
| | 2 | 2.2 × 10⁵ | 0.4 | 3.6 × 10⁴ | 1.2 | 4.0 × 10³ | 2.2 |
| 100% | 1 | 1.2 × 10⁵ | 0.7 | 1.2 × 10⁵ | 0.7 | 1.1 × 10² | 0.8 |
| Hibiclens | | | | | | | |
| | 2 | 3.2 × 10⁵ | 0.3 | 3.4 × 10⁵ | 0.3 | 9.1 × 10⁴ | 0.3 |
| 100% Betadine | 1 | 2.2 × 10⁴ | 1.5 | 2.5 × 10³ | 2.4 | 1.9 × 10² | 3.5 |
| | 2 | 2.1 × 10⁴ | 1.5 | 2.0 × 10³ | 2.5 | 1.9 × 10² | 3.5 |

TABLE 16b

| Test Agent | Rep | 2 hr | LR | 3 hr | LR | 24 hr | LR |
|---|---|---|---|---|---|---|---|
| 20% AMC | 1 | 3.2 × 10³ | 2.3 | 2.5 × 10³ | 2.4 | 2.3 × 10² | 3.4 |
| | 2 | 2.3 × 10³ | 2.4 | 2.2 × 10³ | 2.5 | 5.0 × 10² | 3.1 |
| 100% | 1 | 5.7 × 10² | 3.0 | 6.0 × 10¹ | 4.0 | 1.0 × 10⁰ | 5.8 |
| Hibiclens | | | | | | | |
| | 2 | 6.1 × 10² | 3.0 | 9.0 × 10¹ | 3.8 | 1.0 × 10⁰ | 5.8 |
| 100% Betadine | 1 | 1.1 × 10⁰ | 5.8 | 1.0 × 10⁰ | 5.8 | 1.0 × 10⁰ | 5.8 |
| | 2 | 1.0 × 10⁰ | 5.8 | 1.0 × 10⁰ | 5.8 | 1.0 × 10⁰ | 5.8 |

### Example 10

### Testing the Disinfecting Property of AMC Against Select Bacteria, Yeast, and Fungi

### I. General Procedures for MIC, MBC and MFC Testing

A. Bacterial, Yeast, and Fungal Strains and Culture Conditions

Strains of E. coli (ATCC 11229), D. aeruginosa (ATCC 15442), S. aureus (ATCC 25923), S. epidermidis (ATCC 12228), S. pyogenes (ATCC 19615), C. albicans (ATCC 10231), and T. rubrum (ATCC 28188) were used as “challenge microorganisms” in this study. Strains were removed from frozen stock and subcultured twice on blood agar to ensure that the strains had optimal growth and metabolic status before testing.

B. Broth Microdilution

Strains were tested by NCCLS broth microdilution (M7-A6, 2003 [bacteria]; M27-A2, 2002 [fungi]) and challenged with the AMC solution and its individual components ZnEDTA, CPC (CPC), and isopropanol. Hibiclens (industry comparator) and ciprofloxacin were added to the bacterial panel. Amphotericin B, fluconazole, and Hibiclens were added as comparative agents in the fungal panels.

C. Microtiter Panels for Broth Microdilution Testing

AMC, ZnEDTA, CPC, and isopropanol were provided by DPT Laboratories (Texas, USA). Upon receipt, AMC components were diluted to a working solution equal to their concentration in 100% AMC, i.e., 5% ZnEDTA; 1% CPC; and 49% isopropanol. For testing purposes, AMC, AMC components, and Hibiclens were diluted to 50% of the working solution to cover the proposed AMC use dilution of 20%.

Microtiter panels were created on the day of MIC testing. A 50 μl aliquot of Mueller-Hinton broth (for bacterial strains) or RPMI 1640 (for fungal strains) was delivered to each well, except those in column one of the microtiter panel. The working solution of AMC and each of its components, and comparative agents were delivered to the designated wells in column one. AMC, components, and comparators were serially diluted across the panel. A 50 μl aliquot of bacterial or fungal suspension was then added to each well on the microtiter panel, using a new pipette tip for each aliquot. Panels were incubated at 35° C. for 16-20 h for the E. coli, P. aeruginosa, and S. aureus strains, 20-24 h for the S. pyogenes strain, and 48 h for the C. albicans strain. T. rubrum panels were incubated for 3 to 4 days before MICs were read. Bacterial MICs (%) were read using the lowest antimicrobial concentration that completely inhibited bacterial growth. Fungal MICs (%) were read using an 80% reduction in the growth endpoint relative to the turbidity of the growth control for AMC, its components, and fluconazole. For amphotericin B, the endpoint was read as the complete inhibition of growth.

AMC, component, and comparator panel ranges for the present study are summarized in Table 17.

TABLE 17

| Agent | Range |
|---|---|
| AMC | 0.05-50.0% |
| Zn/EDTA | 0.0025-2.5% |
| Cetylpyridium chloride | 0.007-0.5% |
| Isopropanol | 0.025-24.5% |
| Hibiclens | 0.05-50.0% |
| Ciprofloxacin | 0.03-32 μg/ml |
| Fluconazole | 0.25-256 μg/ml |
| Amphotericin B | 0.008-8.0 μg/ml |

D. MBC and MFC determinations

MBC determinations were performed in accordance with published NCCLS methods (M26-A, 1999) to determine the concentration at which ≧99.9% of the starting bacterial inoculum was killed. MBCs were determined for each strain by culturing 10 μl of each dilution well that showed no visible bacterial growth when the MIC was read. The 10 μl samples were plated on to blood agar and incubated at 35° C. for 24 h (E. coli and P. aeruginosa) or 48 h (S. aureus, S. epidermidis, and S. pyogenes). After incubation, colony counts for each panel well plated were recorded. The 10 μl aliquot of the lowest antimicrobial concentration that demonstrates a kill level of ≧99.9% relative to the starting inoculum was considered the MBC.

MFCs were determined in a similar manner. 10 μl samples of each dilution well above the MIC were plated to Sabouraud dextrose agar and incubated at 35° C. for 24 h (C. albicans) or 72 h (T. rubrum). The MFC was determined as the lowest concentration at which there was a 99.9% reduction in CFU/ml compared with the original organism concentration (1×10³ cells/ml).

### II. Experimental Results

MICs (minimum inhibitory concentration) and MBCs (minimum bactericidal concentration) of five bacterial ATCC strains (Table 18) and MICs and MFCs (minimum fungicidal concentration) for two fungal ATCC strains (Table 19) were tested against the novel solution AMC, its components, and comparative agents.

As shown in Table 18, the minimum inhibitory concentration (MIC) is the lowest concentration that completely inhibits bacterial growth. The MIC % is expressed as g/100 ml or ml/100 ml. The minimum bactericidal concentration (MBC) is the lowest antimicrobial concentration that completely inhibits bacterial growth. The MBC % is expressed as g/100 ml or ml/100 ml. The components of the AMC preparation are in the following proportions: ZnEDTA, 5%; CPC, 1%; Isopropanol, 49%; water. 45%.

TABLE 18
MIC and MBC of AMC, individual AMC components, and
comparator compounds for select bacteria

| | MIC, % | | | | | | | | | |
|---|---|---|---|---|---|---|---|---|---|---|
| | S. epidermidis | | S. aureus | | S. pyogenes | | E. coli | | P. aeruginosa | |
| | ATCC 12228 | | ATCC 25923 | | ATCC 19615 | | ATCC 11229 | | ATCC 15442 | |
| Compound | MIC | MCB | MIC | MBC | MIC | MBC | MIC | MBC | MIC | MBC |
| AMC | <=0.05 | <=0.05 | <=0.05 | <=0.05 | <=0.05 | <=0.05 | 0.2 | ND | 1.6 | 1.6 |
| ZnEDTA (μg/ml) | 1.3(13000) | >2.5 | 1.3 | >2.5 | 1.3(13000) | 1.3 | 2(20000) | ND | 2(20000) | 8(80000) |
| | | (>25000) | (13000) | (>25000) | | (13000) | | | | |
| Cetylpyridinium | <=0.0007 | <=0.0007 | <=0.0007 | <=0.0007 | <=0.0007 | <=0.0007 | 0.005 | ND | 0.02 | 0.04 |
| chloride (μg/ml) | (<=7) | (<=7) | (<=7) | (<=7) | (<=7) | (<=7) | (50) | | (200) | (400) |
| Isopropanol | 12.3 | 24.5 | 12.3 | 24.6 | 12.3 | 12.3 | 6.1 | ND | 3.1 | 12.3 |
| Hibiclens | <=0.05 | <=0.05 | <=0.05 | <=0.05 | <=0.05 | <=0.05 | <=0.05 | ND | <=0.05 | <=0.05 |
| Ciprofloxacin, | 0.25 | 0.25 | 0.5 | 0.5 | 0.5 | 0.5 | <=0.03 | ND | 0.12 | 0.5 |
| μg/ml | | | | | | | | | | |

AMC, at a concentration of ≦0.05%, inhibited the growth of S. aureus ATCC 25923, S. pyogenes ATCC 19615, and S. epidermidis ATCC 12228. Among these strains, CPC (≦0.0007%, ≦7 μg/ml) was the most active component of AMC. AMC also showed activity against E. coli ATCC 11229 (0.2%) and P. aeruginosa ATCC 15442 (1.6%); CPC was the most active component of AMC (0.005%, 50 μg/ml; 0.02%, 200 μg/ml, respectively) tested against these strains. The comparator, Hibiclens, demonstrated consistent activity (≦0.05%) against all five bacterial ATCC strains tested. For S. aureus ATCC 25923, S. epidermidis ATCC 12228, and P. aeruginosa ATCC 15442 isolates, AMC and its components CPC and isopropanol, and comparators Hibiclens and ciprofloxacin showed bactericidal activity (i.e., an MBC within one doubling dilution of the MIC). AMC and all its components, as well as the comparator compounds, were bactericidal for S. pyogenes ATCC 19615. Bactericidal activity was also observed for AMC, cetylpyridinium, and Hibiclens against P. aeruginosa ATCC 15442; ZnEDTA, isopropanol, and ciprofloxacin MBC results were >1 doubling dilution higher than the MIC results and therefore did not meet the definition of bactericidal (Table 18).

As shown in Table 19, the MIC was read as an 80% reduction in growth endpoint relative to the turbidity of the growth control for AMC, AMC components, Hibiclens, and fluconazole. The endpoint for amphotericin B was read as complete inhibition of growth. The MIC % is expressed as g/100 ml or ml/100 ml. The minimum fungal concentration (MFC) is the lowest concentration at which there was a 99.9% inhibition of growth. The MFC % is expressed as g/100 ml or ml/100 ml. The components of the AMC preparation are in the following proportions: ZnEDTA, 5%; CPC, 1%; Isopropanol, 49%; water, 45%.

TABLE 19

| | MIC, % | | | |
|---|---|---|---|---|
| | C. albicans ATCC 1D231 | | T. rubrum ATCC 28188 | |
| Compound | MIC | MFC | MIC | MFC |
| AMC | <=0.05 | <=0.05 | <=0.05 | <=0.05 |
| ZnEDTA(μg/ml) | 0.15 (1500) | 0.15 (1500) | 0.08 (800) | 0.3 (3000) |
| CPC (μg/ml) | <=0.0007 (<=7) | <=0.0007 (<=7) | <=0.0007 (<=7) | <=0.0007 (<=7) |
| Isopropanol | 1.5 | 1.5 | 0.2 | 0.4 |
| Hibiclens | <=0.05 | <=0.05 | <=0.05 | <=0.05 |
| Fluconazole, | 0.5 | 2 | 2 | 8 |
| μg/ml | | | | |
| Amphotericin B, | 0.12 | 0.12 | 0.25 | 0.5 |
| μg/ml | | | | |

For C. albicans ATCC 10231, AMC had an MIC of ≦0.05% CPC was the most active component of AMC with an MIC ≦0.0007% (≦7 μg/ml). The industry comparator, Hibiclens, also demonstrated good activity against C. albicans ATCC 10231 (MIC, ≦0.05%). Fungal activity, defined by an MFC within 1 doubling dilution of the MIC, was observed for AMC and its components ZnEDTA, CPC, and isopropanol, and comparators Hibiclens and amphotericin B. The fluconazole MFC result was >1 doubling dilution higher than the MIC result and was therefore not considered fungicidal (Table 19).

For T. rubrum ATCC 28188, AMC had an MIC of ≦0.05%; CPC was the most active component of AMC (MIC, ≦0.007%, ≦7 μg/ml). The comparator, Hibiclens, also demonstrated an MIC of ≦0.05%. Fungicidal activity was observed for AMC and its components cetylpyridinium chloride and isopropanol, Hibiclens and amphotericin B. ZnEDTA and fluconazole did not demonstrate fungicidal activity (Table 19).

AMC demonstrated more potent in vitro activity against the three ATCC strains of Gram-positive bacteria and fungi (MICs, ≦0.05%) than against Gram-negative bacteria (MICs, 0.2-1.6%). CPC was the most active component of AMC. AMC and CPC demonstrated bactericidal activity against both Gram-positive and Gram-negative ATCC strains and fungicidal activity against ATCC strains of C. albicans and T. rubrum. AMC was as active as Hibiclens against the three Gram-positive ATCC strains, C. albicans ATCC 10231 and T. rubrum ATCC 28188, but was less active against E. coli ATCC 11229 and P. aeruginosa ATCC 15442 than was Hibiclens.

### Example 11

### Testing of the Disinfecting Property of AMC Against a Yeast and a Fungus

### I. General Procedures for MIC, MBC and MFC Determinations

A. Fungal Strains and Culture Conditions

Strains of C. albicans (ATCC 10231) and T. rubrum (ATCC 28188) were tested in this study. Strains were removed from frozen stock and subcultured twice on blood agar to ensure that the strains had optimal growth and metabolic status before testing.

B. Broth Microdilution Testing

Strains were tested by NCCLS broth microdilution (M27-A2, 2002) and challenged with AMC solution and its individual components ZNEDTA, CPC (CPC), and isopropanol. Amphotericin B, fluconazole, terbinafine, and Hibiclens were added as comparator compounds.

C. Microtiter Panels for Broth and Microdilution Testing

AMC, ZnEDTA, CPC, and isopropanol were provided by DPT Laboratories (Texas, USA). Upon receipt, AMC components were diluted to a working solution equal to their concentration in 100% AMC, i.e., 5% ZnEDTA; 1% CPC; and 49% isopropanol. For testing purposes, AMC, AMC components, and Hibiclens were diluted to 50% of the working solution to cover the proposed AMC use dilution of 20%.

Microtiter panels were created on the day of MIC testing. A 50 μl aliquot of RPMI 1640 was delivered to each well except those in column one of the microtiter panel. The working solution of AMC and each of its components, and comparative agents were delivered to the designated wells in column one; AMC, components, and comparators were serially diluted across the panel. A 50 μl aliquot of fungal suspension was then added to each well on the microtiter panel, using a new pipette tip for each aliquot. Panels were incubated at 35° C. for 48 h for the C. albicans strain; T. rubrum panels were incubated for 3 to 4 d before MICs were read. Fungal MICs (%) were read using an 80% reduction in the growth endpoint relative to the turbidity of the growth control for AMC, its components, and fluconazole. For amphotericin B, the endpoint was read as the complete inhibition of growth. Terbinafine MICs were read using a 50% and 90% reduction in the growth endpoint relative to the turbidity of the growth control.

AMC, component, and comparator panel ranges for the present study are summarized in Table 20.

TABLE 20

| Agent | Range |
|---|---|
| AMC | 0.05-50.0% |
| Zn/EDTA | 0.0025-2.5% |
| Cetylpyridium chloride | 0.007-0.5% |
| Isopropanol | 0.025-24.5% |
| Hibiclens | 0.05-50.0% |
| Ciprofloxacin | 0.03-32 μg/ml |
| Fluconazole | 0.25-256 μg/ml |
| Amphotericin B | 0.008-8.0 μg/ml |
| Terbinafine | 0.004-8.0 μg/ml |

D. MFC Determinations

MFCs were performed to determine the concentration at which ≧99% of the starting fungal inoculum was killed. MFCs were determined for each strain by culturing 100 μl (entire content of well) of each dilution well above the MIC. The 100 μl samples were plated on to Sabouraud dextrose agar and incubated at 35° C. for 24 h (C. albicans) or 72 h (T. rubrum). After incubation, colony counts for each panel well plated were recorded. The MFC was determined as the well that displayed no growth (i.e., 99% reduction in CFU/ml compared with the original organism concentration of 1×10³ cells/ml).

### II. Experimental Results

The MIC and MFC for two fungal ATCC strains (Table 21) were tested against the novel AMC solution, its components, and comparative agents.

As shown in Table 21, the MIC was read as an 80% reduction in growth endpoint relative to the turbidity for the growth control for AMC, AMC components, Hibiclens, and fluconazole. The endpoint for amphotericin B was read as complete inhibition of growth. The MIC % is expressed as g/100 ml or ml/100 ml. The minimum fungal concentration (MFC) is the lowest concentration at which there was a 99.9% inhibition of growth. The MFC % is expressed as g/100 ml or ml/100 ml. The components of the AMC preparation were in the following proportions: ZnEDTA, 5%; CPC, 1%; Isopropanol, 49%; water, 45%. Terbinafine MICs were read at both 50% and 90% inhibition of growth (50%/90%).

TABLE 21

| | MIC, % | | | |
|---|---|---|---|---|
| | C. albicans ATCC 10231 | | T. rubrum ATCC 28188 | |
| Compound | MIC | MFC | MIC | MFC |
| AMC | <=0.05 | <=0.05 | <=0.05 | <=0.05 |
| ZnEDTA(μg/ml) | 0.08 (800) | 1.3 (13000) | 0.04 (400) | 0.6 (6000) |
| CPC (μg/ml) | ≦0.0007 (≦7) | ≦0.0007 (≦7) | ≦0.0007 (≦7) | ≦0.0007 (≦7) |
| Isopropanol | 0.8 | 1.5 | 0.2 | 3.1 |
| Hibiclens | ≦0.05 | ≦0.05 | ≦0.05 | ≦0.05 |
| Fluconazole (μg/ml) | 0.5 | 1 | 2 | 64 |
| Amphotericin B (μg/ml) | 0.25 | 0.5 | 0.25 | 0.5 |
| Terbinafine (μg/ml) | >0.5 | >0.5 | 0.008/0.032 | 0.125 |

For C. albicans ATCC 10231, AMC had an MIC of ≦0.05%. CPC was the most active component of AMC with an MIC ≦0.0007% (≦7 μg/ml). The industry comparator, Hibiclens, also demonstrated good activity against C. albicans ATCC 10231 (MIC ≦0.05%). Fungicidal activity, defined by an MFC within 1 doubling dilution of the MIC, was observed for AMC and its components CPC and isopropanol, and comparators Hibiclens, fluconazole, amphotericin B, and terbinafine (Table 21).

For T. rubrum ATCC 28188, AMC had an MIC of ≦0.05%. CPC was the most active component of AMC (MIC ≦0.007%, ≦7 μg/ml). The comparator, Hibiclens, also demonstrated an MIC of 0.05%. Fungicidal activity was observed for AMC and its component CPC, Hibiclens and amphotericin B. ZnEDTA, isopropanol, fluconazole, and terbinafine did not demonstrate fungicidal activity (Table 21).

AMC demonstrated potent in vitro activity against C. albicans ATCC 10231 and T. rubrum ATCC 28188 (MICs, ≦0.05%). CPC was the most active component of AMC. AMC and CPC demonstrated fungicidal activity against ATCC strains of C. albicans and T. rubrum. AMC was as active as Hibiclens against the two fungal ATCC strains. Among the antifungal agents tested, terbinafine was the most active agent (MIC 0.008 μg/ml) tested against T. rubrum ATCC 28188, but the least active antifungal agent (MIC >0.5 μg/ml) tested against C. albicans ATCC 10231.

### Example 12

### Synergy Between the Components of the Microbiocidal/Sporicidal Composition

The antifungal action of AMC against the tested fungal species is not a characteristic of either CPC-based, IPA-based, or Zn-based antimicrobial products, at the CPC, IPA, or Zn concentrations involved, and appears to result from the synergistic interaction of the active ingredients. This is easily seen from data on killing of Trichophyton rubrum in which various CPC concentrations were tested in the presence of either Zn⁺² (as Zn-EDTA) or IPA or both. This is summarized in Table 22 below.

TABLE 22
Synergy Between AMC Components as Demonstrated by 30-Sec
Killing of Trichophyton rubrum (2,200,000 CFU initial)

| | 30-Second Reduction | Relative Kill |
|---|---|---|
| Component or Mixture | from Initial CFU Values | Potency |
| 0.002% CPC | 9.36-fold (0.97-logs) | 1.00 |
| 9.8% IPA | 5.95-fold (0.77-logs) | 0.64 |
| 1% Zn-EDTA | 4.89-fold (0.69-logs) | 0.52 |
| 0.002% CPC + 9.8% IPA | 6.38-fold (0.80-logs) | 0.68 |
| 1% Zn-EDTA + 0.1% CPC | 10.0-fold (1.0-logs) | 1.06 |
| 0.002% CPC + 1% Zn-EDTA + | 53-fold (1.72-logs) | 5.66 |
| 9.8% IPA | | |

As can be seen from the data in Table 22, only the combination of CPC, IPA, and Zn gives a performance boost compared to any other combination or to the individual active ingredients. The data extracted for Table 22 were for a condition in which killing of the microorganism in question was challenging, as otherwise synergism is more difficult to demonstrate. That is, in the case where 100% killing occurs easily, the performance differences between different combinations of components is masked by the fact one or more of the individual components may give 100% kill also. For AMC acting on bacteria this occurs frequently with the CPC alone.

Studies measuring the MIC (minimum inhibitory concentration) for AMC compared to ZnEDTA, CPC, isopropanol, Hibiclens, and ciprofloxacin (against bacteria) and ZnEDTA, CPC, isopropanol, Hibiclens, fluconazole, and amphotericin B (against yeast) show that AMC has excellent potency against the tested organisms and in relation to the comparison antimicrobials tested. The MIC is the lowest antimicrobial concentration which completely inhibits growth. These data are summarized in Table 23 and Table 24 below. In these tables, a lower MIC corresponds to a higher potency.

TABLE 23
MIC values for AMC and Comparison Products/Materials

| | MIC Against - | | | | | | |
|---|---|---|---|---|---|---|---|
| Test Material | S. epidermidis | S. aureus | S. pyogenes | E. coli | P. aeruginosa | C. albicans | T. rubrum |
| AMC (ppm) | 0.016 | 0.008 | 0.016 | 0.25 | 2.0 | 0.031 | 0.031 |
| Hibiclens | 3.91 | <1.95 | 3.91 | 3.91 | 125 | 7.81 | 31.25 |
| (ppm) | | | | | | | |
| Ciprofloxacin | >0.1 | >0.1 | >0.1 | >0.1 | >0.1 | >0.1 | >0.1 |
| (ppm) | | | | | | | |
| Miconazole | n.d. | n.d. | n.d. | n.d. | n.d. | 2.0 | 0.25 |
| (ppm) | | | | | | | |
| Amphotericin B | n.d. | n.d. | n.d. | n.d. | n.d. | n.d. | >0.25 |
| (μg/mL) | | | | | | | |

TABLE 24
MIC values for AMC and Comparison Products/Materials

| | MIC Against - | | | | | | |
|---|---|---|---|---|---|---|---|
| Test Material | S. epidermidis | S. aureus | S. pyogenes | E. coli | P. aeruginosa | C. albicans | T. rubrum |
| AMC (%) | ≦0.05 | ≦0.05 | ≦0.05 | 0.2 | 1.6 | ≦0.05 | ≦0.05 |
| AMC Al* | ≦5 | ≦5 | ≦5 | 30 | 160 | ≦5 | ≦5 |
| (μg/mL) | | | | | | | |
| ZnEDTA | 13,000 | 13,000 | 13,000 | 13,000 | 20,000 | 800 | 400 |
| (μg/mL) | | | | | | | |
| CPC** | ≦7 | ≦7 | ≦7 | 50 | 200 | ≦7 | ≦7 |
| (μg/mL) | | | | | | | |
| IPA (%) | 12.3 | 12.3 | 12.3 | 6.1 | 3.1 | 0.8 | 0.2 |
| Hibiclens (%) | ≦0.05 | ≦0.05 | ≦0.05 | ≦0.05 | ≦0.05 | ≦0.05 | ≦0.05 |
| Ciprofloxacin | 0.25 | 0.5 | 0.5 | ≦0.03 | 0.12 | | |
| (μg/mL) | | | | | | | |
| Fluconazole | | | | | | 0.5 | 2 |
| (μg/mL) | | | | | | | |
| Terbinafine | | | | | | >0.5 | 0.008 |
| (μg/mL) | | | | | | | |
| Amphotericin | | | | | | 0.25 | 0.25 |
| B (μg/mL) | | | | | | | |

### Example 13

### Pharmacology, Microbiology, Toxicology and Safety of Isopropanol, Zinc, and CPC

### I. Pharmacology & Microbiology

Relevant pharmacology and microbiology for AMC's active ingredients is discussed below.

A. Isopropanol

Isopropanol (IPA), like any aliphatic alcohol, has central nervous system depressant properties and can modulate the liver toxicity of other compounds. It is irritant to the eyes and mucous membranes. It is reported to induce mixed function oxidases of the liver.

Isopropanol is bactericidal and is used in a number of products such as topical antiseptics, disinfectants for home, hospital, and industry, rubbing alcohol, medicinal liniments, tinctures of green soap scalp tonics tincture of mercurophen, and pharmaceuticals (e.g., local anesthetics, tincture of iodine, and bathing solutions for surgical sutures and dressings). Kirk-Othmer Encyclopedia of Chemical Technology. 4th ed. Volumes 1: New York, N.Y. John Wiley and Sons, 1991-Present, p. V20 (1996) 236. Isopropanol is also used as a skin wipe applied to reduce local bacterial flora prior to penetration with needles or other sharp instruments and also as a preoperative wash. Isopropyl alcohol has slightly greater bactericidal activity than ethyl alcohol due to its greater depression of surface tension. It rapidly kills vegetative forms of most bacteria when used full strength or as 70% aqueous solution. American Medical Association, Department of Drugs. Drug Evaluations. 6th ed. Chicago, Ill: American Medical Association, 1986, 1523. Viricidal activity has also been reported: In an animal study Hepatitis B virus in dried human plasma was exposed for 10 min at 20 deg C. to 70% isopropanol. One chimpanzee received the treated viral material intravenously, and did not show signs of infection over a post-inoculation period of 9 months. Bond W W. et al., J Clin Microbial 18 (3): 535 (1983).

A traditional weakness of isopropanol as a stand-alone is that it has essentially no useful effect against fungi. This weakness is eliminated in the AMC formulation. This is shown in Table 25 below, which compares the effectiveness of 9.8% IPA against T. rubrum and C. albicans with the effectiveness of a 20% (1:5) AMC dilution (containing 9.8% IPA) and of the other AMC components at the concentrations present in a 20% AMC dilution.

TABLE 25
Anti-Fungal Effectiveness of 20% AMC vs. Equivalent % IPA
and other AMC ™ Components as Demonstrated by
30-Sec Killing of T. rubrum and C. albicans

| | 30-Second | 30-Second Log |
|---|---|---|
| | Log Reduction from | Reduction from Initial |
| | Initial T. rubrum | T. rubrum CFU |
| Component or Mixture | CFU Values | Values |
| AMC ™, 1:5 dilution | 6.3 Logs (100% Kill) | 6.9 Logs (100% Kill) |
| (20%) | | |
| 9.8% IPA (=20% | 0.8 Logs | 0.3 Logs |
| AMC ™) | | |
| 0.2% CPC (=20% | 3.4 Logs | 6.9 Logs (100% Kill) |
| AMC) | | |
| 1% Zn-EDTA (=20% | 0.7 Logs | 0.1 Logs |
| AMC) | | |

B. Zinc (As the Oxide, Salts, or Chelated Complexes Such as with EDTA)

Zinc is an essential nutrient mineral and functions as a co-factor for some enzymes. Dietary deficiency results in severe health consequences. Overexposure to zinc requires a very substantial exposure (excepting exposure to zinc metal fumes, see zinc toxicology in next subsection) and is unusual. Zinc does not accumulate in the body. Average adult daily intake in the U.S. is 12-15 mg daily, mostly from foods. Goyer, R. A., “Toxic Effects of Metals,” Chapt. 23 in Casarett and Doull's Toxicology, 5th Ed. (1996), pp. 720-721, McGraw-Hill, NY.

As the oxide, other zinc salts, or a chelated zinc, zinc has mild astringent and antiseptic action and is used in the treatment of skin diseases and infections such as eczema, impetigo, ringworm, varicose ulcers, pruritus, and psoriasis. Zinc-undecylenate (salt of C11-fatty acid, also called zinc-undecate) is a common OTC product for athlete's foot and other dermatomycoses (as a cream) and for treatment of styes (as an ointment). Zinc oxide paste with salicylic acid NF is frequently used in treatment of athlete's foot and other dermatomycoses. The presence of zinc oxide imparts astringent and protective property to the paste. Astringent action is desired to reduce inflammation and to close fissures. Gilman, A. G., L. S. Goodman, and A. Gilman (eds.) Goodman and Gilman's The Pharmacological Basis of Therapeutics. 7th ed. New York: Macmillan Publishing Co., Inc., 1985. 967.

In summary, zinc is used pharmacologically for its antiseptic properties and for astringency in dermal preparations. In AMC, the combination of ingredients does require zinc for the full synergy of the product. A much higher biocidal activity and breadth of efficacy is observed with the AMC solution than is seen for zinc itself.

C. Cetylpyridinium Chloride

Cetylpyridinium chloride (CPC) is a cationic surfactant agent. Its pharmacology appears to involve both the CNS and muscarinic receptors in the PNS. Ingestion of large quantities may cause nausea, vomiting, collapse, convulsions and coma as well as, in animal studies, a curare-like transient paralysis of motor function. At the CPC levels used in AMC concentrate, the potential negative pharmacological effects of CPC are not reasonably anticipated to occur.

Cetylpyridinium chloride (CPC) is well known as an antiseptic and antimicrobial and is also used as a preservative for cosmetics and pharmaceuticals. Ashford, R. D. Ashford's Dictionary of Industrial Chemicals. London, England: Wavelength Publications Ltd., 1994. 189. CPC is also the main active ingredient in Cepacol® products, including throat lozenges and mouthwash. Kirk-Othmer Encyclopedia of Chemical Technology. 4th ed. Volumes 1: New York, N.Y. John Wiley and Sons. 1991-Present, p. V8 (1993) 259 & 851. It is also used in external deodorant products.

Its most common medical use is as a local antiinfective that possesses surface-active as well as antiseptic properties against sensitive nonsporulating bacteria. It is frequently used for pre-operative preparation of skin, for prophylactic antisepsis of minor wounds, and for irrigation of or topical application to mucous membranes (for example, by incorporation into mouthwashes). Osol, A. and J. E. Hoover, et al (eds.). Remington's Pharmaceutical Sciences. 15th ed. Easton, Pa.: Mack Publishing Co., 1975. 1090.

Traditionally, a weakness of CPC (and other alkyl/aryl-quaternary ammonium) based antimicrobials has been that, while these are very effectively bactericidal or bacteriostatic against many gram-positive and gram-negative organisms and have some degree of activity against specific fungi (notably Candida albicans and Trichomonas vaginalis), they are not effective against bacterial spores or most viruses. American Hospital Formulary Service, Volumes I and II. Washington, D.C.: American Society of Hospital Pharmacists, to 1984, p. 84:4:16. In contrast to other cationic surfactants, the antibacterial activity of CPC changes little over a pH range of 2 to 10.

Another issue for CPC-based stand alone products is their slow action time. For example, a 0.1% solution applied to human skin will typically require about 7 min to decrease the bacterial population by 50% (i.e., by 2-fold). An 0.1% CPC tincture has slower action than 70% ethanol. Even in the absence of antagonistic tissue constituents, 0.002% CPC solution requires about 9 hr to kill 98% of Escherichia coli (i.e., a 50-fold reduction). Goodman, L. S., and A. Gilman. (eds.) The Pharmacological Basis of Therapeutics. 5th ed. New York. Macmillan Publishing Co., Inc., 1975. 1002.

These traditional CPC weaknesses appear not to be an issue for the AMC technology. The data in Table 1 above establish that bacterial killing by AMC dilutions of even up to 1:500 (0.002% CPC equivalent) achieve 100% kill of E. coli, S. aureus, and P. aeruginosa by 30 seconds. The same data also show that AMC is an effective fungicide and anti-fungal.

For use in medical applications, typical dosages or strengths for CPC in CPC-based products are as per Table 26 below.

TABLE 26
Typical Medicinal Use CPC-Strengths/Dosages

| | | | Other |
|---|---|---|---|
| Route/Form | Target | % CPC Solution | Forms Dose |
| Topical, solution | Intact Skin | 0.1%-1% | |
| | Minor Lacerations | 0.1% | |
| | Mucous Membranes | 0.01%-0.05% | |
| Lozenge, Troche | Mucous Membranes | 0.01%-0.05% | 0.33-3 mg |
| | | | (0.067%) |
| Lavage solution | Rectum | 0.05% | |
| Suppository | Vagina (vaginitis) | | 0.1% |

Sources for information summarized contained in Table 26 are Osol, A. and J. E. Hoover, et al. (eds.). Remington's Pharmaceutical; Sciences. 15th ed. Easton, Pa.: Mack Publishing Co., 1975. 1090, and American Hospital Formulary Service. Volumes I and II. Washington. D.C.: American Society of Hospital Pharmacists, to 1984, p. 84:4:16

In dental hygiene applications, it is reported that plaque provoked by sucrose rinses was moderately inhibited by quaternary ammonium compounds (including cetylpyridinium chloride) rinsing twice daily with 2.2 mMolar CPC (0.075%) and that 4 times/day rinsing increased the effectiveness to almost that of chlorhexidine. Bonesvoll P, Gjermo P; Arch Oral Biol 23 (4): 289-94 (1978).

The ability of AMC to provide effective control of both bacteria and fungi, at lower overall CPC (and associated IPA, and Zn) levels than are traditionally required and to provide fungal control, not typically seen with either IPA or CPC based products, opens up a number of potential medical and dental applications for the AMC technology in which both unit cost for the active ingredients, breadth of antimicrobial spectrum, and lowered potential for local tissue irritation become competitive factors for AMC.

### II. Toxicology and Safety

AMC concentrate has an acute toxicity profile that would be considered as very low acute toxicity. The following toxicity index values have been determined for AMC:

| | |
|---|---|
| Rat oral LD₅₀ | >5,000 mg/Kg |
| Rabbit dermal LD₅₀ | >5,000 mg/Kg |
| Rat 4 h inhalation LC₅₀ | >2 mg/L |
| Eye irritation | Severe Irr. (Cat I-II) |
| Dermal irritation | Mod. Irr. (Cat III) |
| Dermal sensitization | Non-sensitizing |

Due to the IPA content and the CPC content of the concentrate, AMC concentrate produces severe eye irritation. AMC concentrate produces only moderate skin irritation. These effects will be diminished for dilutions of AMC and for AMC formulations or AMC-treated products in which AMC is reduced in overall concentration compared to AMC concentrate itself. It is anticipated that products derived from AMC concentrate will have little potential for eye or skin irritation due to the degree of concentration reduction for various dilutions of AMC (see Table 26). The information in Table 27 suggests that dilutions of 1:50 and higher should have essentially no eye or skin irritancy and that the 1:5 dilution will be only moderately irritating to the eye and essentially non-irritating to the skin.

TABLE 27
Component Concentrations in Various Strengths of AMC

| | | | | 1:500 |
|---|---|---|---|---|
| | Full | 1:5 Dilution | 1:50 Dilution | Dilution |
| Component | Strength | (20%) | (2%) | (0.2%) |
| Isopropanol | 49.00% | 9.80% | 0.98% | 0.098% |
| (Active) | | 98,000 ppm | 9,800 ppm | 980 ppm |
| CPC (Active) | 1.00% | 0.20% | 0.020% | 0.0020% |
| | | 2,000 ppm | 200 ppm | 20 ppm |
| Zn, as oxide | 0.12% | 0.024% | 0.0024% | 0.00024% |
| (Active) | | 240 ppm | 24 ppm | 2.4 ppm |
| EDTA (Inert: | 0.47% | 0.047% | 0.0047% | 0.00047% |
| buffer, chelator) | | 470 ppm | 47 ppm | 4.7 ppm |

The acute toxicity profile for AMC is consistent with the individual acute toxicities of its components and there is no suggestion of synergism of the toxicity of the AMC components when combined into AMC. This can be seen in Table 28 below.

TABLE 28
Component Toxicity Indices Compared to AMC Data

| Material | Rat Oral LD₅₀ | | Rabbit Dermal LD₅₀ | | Rat Inhalation LC₅₀ | |
|---|---|---|---|---|---|---|
| Isopropanol (100%) | >5,000 | mg/Kg | 12,800 | mg/Kg | 72.6 | mg/L |
| CPC (100%) | 200 | mg/Kg | >2,000 | mg/Kg | 0.090 | mg/L |
| Zn oxide (100%) | 7,950 | mg/Kg* | >2,000 | mg/Kg | >0.005 | mg/L |
| EDTA (100%) | 30 | mg/Kg* | 300 | mg/Kg*** | 0.4 | mg/L*** |

Estimated for AMC contained concentrations

| | | | | | | |
|---|---|---|---|---|---|---|
| Isopropanol (49%) | >5,000 | mg/Kg** | 25,600 | mg/Kg | 145 | mg/L |
| CPC (1%) | 20,000 | mg/Kg | >20,000 | mg/Kg** | 9 | mg/L |
| Zn oxide (0.12%) | >>100,000 | mg/Kg | >>100,000 | mg/Kg | >4.2 | mg/L** |
| EDTA (0.47%) | 6,300 | mg/Kg | 64,000 | mg/Kg*** | 11 | mg/L*** |
| Actual Data for AMC | >5,000 | mg/Kg | >5,000 | mg/Kg | >2 | mg/L |
| (100%) | | | | | | |

*Mouse data;
**Index component for toxicity by specific route;
***Estimated from oral toxicity on basis of 10% dermal uptake and 4 hr rat inhalation exposure of 350 g rats with 0.1 L/min minute-volume.

### Equivalents

From the foregoing detailed description of the specific embodiments of the invention, it should be apparent that unique bioactive compositions have been described. Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. For instance, the choice of the route of administration is believed to be a routine matter for a person of ordinary skill in the art with knowledge of the embodiments described herein.

1. A pharmaceutical composition comprising:
a) an ionogenic surfactant;
b) a metal chelating complex comprising a metal and a monodentate, bidentate, or polydentate ligand that exhibits affinity for hydrogen ion;
c) a solvent; and,
d) a pharmaceutically acceptable carrier.

2. The pharmaceutical composition of claim 1, wherein said composition comprises by weight about 0.1-15% ionogenic surfactant, about 1-30% metal chelating complex, and about 0.5-95% solvent.

3. The pharmaceutical composition claim of 1, wherein said metal is selected from the group consisting of copper, zinc, mercury, chromium, manganese, nickel, cadmium, arsenic, cobalt, aluminum, lead, selenium, platinum, gold, titanium, tin, and combinations thereof.

4. The pharmaceutical composition of claim 1, wherein the monodentate, bidentate, or polydentate ligand is selected from the group consisting of anions of natural amino acids, iminodiacetic acids, nitriletriacetitic acids, derivatives of iminodiacetic acids comprising a carbon-substitution in the α-position to the carboxylic group and amino acid residue fragments containing no aminocarboxylic group, derivatives of nitriletriacetic acids comprising a carbon-substitution in the α-position to the carboxylic group and amino acid residue fragments containing no aminocarboxylic group, alkylenediaminopolyacetic acid, derivatives of polyalkylenepolyaminopolyacetic acids comprising a carbon-substitution in the α-position to the carboxylic group and amino acid residue fragments containing no aminocarboxylic group, derivatives of ω-phosphoncarboxylic, derivatives of ethylenediphosphontetrapropionic acids, derivatives of ethylenetetra(thioacetic), derivatives of diethylenetrithiodiacetic acids, monoamine complexones in which carboxylic groups are replaced by phosphonic groups, and mixtures thereof.

5. The pharmaceutical composition of claim 1, wherein the metal chelating complex comprises at least one amino acid selected from the group consisting of isoleucine, phenylalanine, leucine, lysine, methionine, threonine, tryptophan, valine, alanine, glycine, arginine, and histidine.

6. The pharmaceutical composition of claim 1, wherein the metal chelating complex comprises a glycinatecopper halide complex.

7. The pharmaceutical composition of claim 1, wherein the metal chelating complex comprises an ethylenediaminotetraacetate zinc (Zn-EDTA) complex.

8. (canceled)

9. (canceled)

10. The pharmaceutical composition of claim 1, wherein the ionogenic surfactant is selected from the group consisting of cetylpyridinium chloride (CPC), cetyltrimethylammonium chloride, cetylbenzyldimethylammonium chloride, cetylpyridinium bromide (CPB), cetyltrimethylammonium bromide (CTAB), cetyldimethylethylammonium bromide, cetyltributylphosphonium bromide, dodecyltrimethylammonium bromide, and tetradecyltrimethylammonium bromide.

11. The pharmaceutical composition of claim 10, wherein the ionogenic surfactant is CPC.

12. The pharmaceutical composition of claim 1, wherein said solvent is an aliphatic alcohol.

13. The pharmaceutical composition of claim 12, wherein said aliphatic alcohol is isopropanol.

14. A The pharmaceutical composition of claim 1, wherein the ionogenic surfactant comprises CPC, the metal chelating complex comprises Zn-EDTA, and the solvent comprises isopropanol.

15. The pharmaceutical composition of claim 14, wherein said composition comprises by weight about 0.2% CPC, about 1% Zn-EDTA, and about 9.8% isopropanol.

16. The pharmaceutical composition of claim 14, wherein said composition comprises by weight about 0.02% CPC, about 1% Zn-EDTA, and about 9.8% isopropanol.

17. The pharmaceutical composition of claim 14, wherein said composition comprises by weight about 0.002% CPC, about 1% Zn-EDTA, and about 9.8% isopropanol.

18. The pharmaceutical composition of claim 1 further comprising at least one additional pharmaceutical composition.

19. The pharmaceutical composition of claim 18, wherein said at least one additional pharmaceutical composition is an antimicrobial composition.

20. The pharmaceutical composition of claim 19, wherein said antimicrobial composition is an antifungal composition.

21. (canceled)

22. The pharmaceutical composition of claim 1, wherein said pharmaceutical composition is formulated for topical administration, oral administration, or intravenous administration.

23. (canceled)

24. (canceled)

25. A method for treating or preventing an infection caused by a pathogen in a subject, said method comprising administering to said subject an effective amount of the pharmaceutical composition of claim 1.

26. The method of claim 25, wherein said pathogen is selected from the group consisting of a bacterium, a virus, and a fungus.

27-36. (canceled)

37. A method for treating or preventing dandruff, acne, or dermatitis in a subject comprising administering to said subject aft effective amount of the pharmaceutical composition of claim 1.

38-40. (canceled)

41. The method of claim 25 further comprising administration of at least one additional pharmaceutical composition.

42-45. (canceled)

46. The method of claim 25, wherein said pharmaceutical composition is administered orally, topically, or intravenously.

47. (canceled)

48. (canceled)
